(19) United States
(12) Patent Application Publication
(10) Pub. No.: US 2018/0353650 A1
(43) Pub. Date: Dec. 13, 2018

(54) BIOCOMPATIBLE MICROFABRICATED MACRODEVICES FOR TRANSPLANTING CELLS

(71) Applicants: Massachusetts Institute of Technology, Cambridge, MA (US); The Children's Medical Center Corporation, Boston, MA (US)

(72) Inventors: Suman Bose, Cambridge, MA (US); Volkan Yesilyurt, Watertown, MA (US); Lisa Rae Volpatti, Cambridge, MA (US); Robert S. Langer, Newton, MA (US); Daniel G. Anderson, Framingham, MA (US)

(21) Appl. No.: 16/007,922

(22) Filed: Jun. 13, 2018

Related U.S. Application Data
(60) Provisional application No. 62/519,020, filed on Jun. 13, 2017.

(51) Int. Cl. A61L 27/38; A61L 27/16; C08L 33/26

(52) U.S. Cl. CPC A61L 27/3834; C08F 20/18; C08L 33/26; A61L 27/16

(57) ABSTRACT

Macrodevices containing a micro-fabricated body having at least one or multiple compartments and a porous membrane, methods of making and using thereof, are described. The one or multiple compartments encapsulate one or more cells that secrete a therapeutic agent in cell-based therapy. The porous membrane provides immunoprotection the encapsulated cells. Further, the surface of the macrodevices is chemically modified using polymers and/or small molecules, reducing fibrosis of the macrodevices, thereby allowing in vivo delivery of the secreted therapeutic agents for extended periods of time.

### CROSS-REFERENCE TO RELATED APPLICATIONS

This application claims benefit of and priority to U.S. Provisional Application No. 62/519,020, filed Jun. 13, 2017, the contents of which is incorporated by reference in its entirety.

### STATEMENT REGARDING FEDERALLY SPONSORED RESEARCH

None.

### FIELD OF THE INVENTION

This invention is in the field of surface-coated macrodevices with a beneficial effect; particularly implantable surface-coated macrodevices containing a chemically modified micro-fabricated body encapsulating cells in a compartment sealed with a porous membrane, for cell-based therapy.

### BACKGROUND OF THE INVENTION

Cell-based therapies have the potential to treat a variety of chronic diseases including diabetes (type 1 and type 2), anemia, liver failure, and Parkinson's disease (Allison, Nat. Rev. Nephrol. 2010, 6, 1-1). The prospect of transplanting cells, such as engineered cells or stem cell-derived cells, that secrete therapeutic agents over long periods of time in cell-based therapies has been an enduring goal. However, foreign implanted cells obtained from other subjects are often immunogenic and are rapidly rejected by the host immune system. Therefore, patients in need of cell transplantation often require systematic immunosuppression for the duration of their lives, which increases the risk of transplant failure, organ damage, and infection (Shapiro, et al., N. Engl. J. Med. 2000, 343, 230-238).

Cell encapsulation provides a safer alternative to immunosuppression for implanting foreign cells in vivo (Nyitray, et al., ACS Nano 2015, 9, 5675-5682; Ludwig, et al., Proc. Natl. Acad. Sci. 2013, 110, 19054-19058). Encapsulation devices function to create an immune-protective environment for the foreign cells that allows for their secretion of therapeutic factors, while maintaining cell viability through effective nutrient and waste exchange after implantation. There are several problems associated with encapsulating foreign cells, including retrievability following implantation, controlling the diffusion of materials (e.g. via control over pore size), biocompatibility, and reproducible fabrication methods (Hunt and Grover, Biotechnol. Lett. 2010, 32, 733-742). Macrodevices have been investigated as possible encapsulation devices. While macrodevices are readily retrievable, they still exhibit problems, such as poor nutrient exchange which leads to necrosis, and prolonged cell response times due to barriers to diffusion. Additionally, macrodevices typically contain sharp corners and rigid structures that lead to host foreign body response and fibrosis, resulting in subsequent device failure.

The development of macrodevices, in particular implantable macrodevices, which permit functional viability of encapsulated cells and/or resist host foreign body response for protracted periods of time is important for improving the performance and safety of such devices, and remains an unmet need. Therefore, the development of encapsulation devices that can encapsulate cells in cell-based therapy, while circumvent the problems associated with currently available implantation devices remains an area of active research.

Therefore, it is an object of the invention to provide macrodevices with improved beneficial effects.

It is another object of the invention to provide macrodevices that maintain functional viability of encapsulated cells and/or resist host foreign body response for protracted periods of time.

It is a further object of the invention to provide macrodevices containing a surface that has been chemically modified.

It is also an object of the invention to provide chemically modified macrodevices that elicit a lower foreign body response and/or maintains improved functional viability of encapsulated cells for protracted periods of time, compared to a corresponding product that lacks the chemical modification.

### SUMMARY OF THE INVENTION

Macrodevices containing a body having one or multiple compartments and a porous membrane sealing the compartments, methods of making and using thereof, are described. Preferably, the body of a macrodevice is formed via micro-fabrication techniques. The one or multiple compartments encapsulate one or more cells that secrete a therapeutic agent in a subject in need thereof post-implantation of the macrodevice. The porous membrane has a pore size that allows diffusion of secreted therapeutic agents, nutrients, oxygen, or combinations thereof, but blocks host immune cells from entering the compartments, thereby providing immunoprotection to the encapsulated cells. Preferably the pore sizes of the porous membrane are 0.8 μm or less.

The macrodevices are superior over current implantation devices in that the micro-fabrication of one or multiple compartments provides precise control over the position of cells relative to the membrane, allowing for sufficient amounts of oxygen and nutrients to reach cells, while also providing easy diffusion of secreted therapeutic agents from the compartments. Further, two or more cells can be placed in close proximity, while remaining physically separated. Preferably, the macrodevices have an oblong shape, rounded corners, or both, and a thickness that is selected to enhance the diffusion of nutrients into and out of a compartment. Preferably, the micro-fabricated body of the macrodevice contains polydimethylsiloxane (PDMS). Preferably, the porous membrane contains polycarbonate. Preferably, the pores are formed by track-etching the polycarbonate membrane. In some forms, the track-etched polycarbonate membrane is attached to one side of the PDMS-containing micro-fabricated body with aminosilane chemistry. Exemplary dimensions of the macrodevice are 1 cm×1.5 cm×1 mm. The first dimension can represent the width, the second dimension the length, and the third dimension the height or thickness of the macrodevice. In some forms, the size of the macrodevice can be scalable based on the desired application.

A surface of the macrodevices can be chemically modified using polymers and/or small molecules. These chemical modifications introduce coatings on the surface of the macrodevice, which reduce fibrosis of the macrodevices, thereby allowing in vivo delivery of therapeutic agents for extended periods of time.

### BRIEF DESCRIPTION OF THE DRAWINGS

FIG. 1 is a schematic of a macrodevice.

FIG. 2 is a line graph showing a human cell line (HEK 293) engineered to secrete a cytokine mouse-erythropoietin (EPO). Cells were encapsulated in macrodevices with different pore sizes and transplanted into the intraperitoneal (IP) space of Balb/c mice. Serum EPO levels were monitored.

FIGS. 3A and 3B show the schematic of chemical modification of a surface of macrodevice by grafting molecules through surface-initiated atom transfer radical polymerization (si-ATRP), FIG. 3A. Monomeric units that can be used in the polymerization reaction are shown in FIG. 3B.

FIG. 4 is a column graph of the X-ray photoelectron spectroscopy (XPS) analysis of the nitrogen content on the surface of an unmodified macrodevice and macrodevice modified, as illustrated in FIG. 3A, using the polymers generated from the monomeric units shown in FIG. 3B.

FIG. 5 is a line graph showing secretion of EPO in mice from the implanted macrodevices described in FIG. 4.

FIGS. 6A and 6B showing protein production of cells implanted with a macrodevice coated with a polymer formed from the monomer (E9, shown in FIG. 3B), for more than 10 weeks in C57BL/6 mice.

FIG. 7 is a column graph showing secretion of EPO in mice from the implanted macrodevices described in FIG. 4 at 4 weeks.

FIG. 8 is a column graph showing the surface concentration of collagen on the macrodevices described in FIG. 4.

FIG. 9 shows the surface DNA content attached to an uncoated and a THPT (E9) coated macrodevices.

FIGS. 10A and 10B are line graphs showing protein production of cells implanted with a macrodevice coated with a polymer formed from the monomer (E9, shown in FIG. 3B), for more than 18 weeks in C57BL/6 mice.

FIG. 11 is a line graph showing serum EPO levels after epo-HEK encapsulated in THPT coated macrodevices were retrieved intact after 75 days.

FIG. 12A and 12B are line graphs showing blood glucose levels and percent cured after rat islets were encapsulated in THPT (E9) coated macrodevices.

### DETAILED DESCRIPTION OF THE INVENTION

I. Definitions

“Biocompatible,” as used herein, refers to a substance or object that performs its desired function when introduced into an organism without inducing significant inflammatory response, immunogenicity, or cytotoxicity to native cells, tissues, or organs. For example, a biocompatible product, e.g., a biocompatible macrodevice, is a product, e.g., a macrodevice, that performs its desired function when introduced into an organism without inducing significant inflammatory response, immunogenicity, or cytotoxicity to native cells, tissues, or organs. Biocompatibility, as used herein, can be quantified using the in vivo biocompatibility assay described below.

In this assay, a material or product can be considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

“Foreign body response” as used herein, refers to the immunological response of biological tissue to the presence of any foreign material in the tissue which can include protein adsorption, macrophages, multinucleated foreign body giant cells, fibroblasts, and angiogenesis.

“Coating” as used herein, refers to any temporary, semi-permanent or permanent layer, covering or surface. A coating can be applied as a gas, vapor, liquid, paste, semi-solid, or solid. In addition, a coating can be applied as a liquid and solidified into a hard coating. Elasticity can be engineered into coatings to accommodate pliability, e.g. swelling or shrinkage, of the substrate or surface to be coated.

“Chemical modification” and related terms, as used herein in the context of the products, refers to chemical modification of the product. “Product” can include devices, such as the macrodevices described herein. Generally, such chemical modification is by direct attachment, coupling, or adherence of a compound to the surface material of the product. Preferably, the chemical modification involves modification with one or more of the compounds. Chemical modification, as defined herein in the context of the products, can be accomplished at any time and in any manner, including, for example, synthesis or production of the modified form of the product or material when the product or material is formed, addition of the chemical modification after the final product or material is formed, or at any time in between. The terms “replaced,” “replace,” “modified,” “singularly modified,” “singular modification,” “multiply modified,” “multiple modifications,” “chemically modified,” “surface modified,” “modification,” “chemical modification,” “surface modification,” “substituted,” “substitution,” “derived from,” “based on,” or “derivatized,” and similar terms, as used herein to describe a structure, do not limit the structure to one made from a specific starting material or by a particular synthetic route. Except where specifically and expressly provided to the contrary, the terms refer to a structural property, regardless of how the structure was formed, and the structure is not limited to a structure made by any specific method.

In some embodiments, where explicitly indicated, addition or application of a material, compound, or composition to a starting material or intermediate before it is made into or incorporated into the final product can be specifically excluded. Thus, for example, chemical modification of alginate or another polymer prior to the polymer being incorporated into a capsule or other structure can be, in some embodiments, specifically excluded as the manner of producing a chemical modification of the capsule or structure. As another example, coating a device, prosthesis, or other product with a material that is chemically modified prior to being applied as a coating can be, in some embodiments, specifically excluded as the manner of producing a chemical modification of the device, prosthesis, or product. However, for such embodiments where such specific exclusions are used, so long as the product was itself chemically modified, coating of or addition to the product of another material that has chemical modifications does not alter the fact that the product was chemically modified according to the meaning of the term used herein.

“Surface modification” and related terms, as used herein in the context of a product, e.g., the macrodevice products, refers to chemical modification of the surface or a surface of the product. Generally, such surface modification is by direct attachment, coupling, or adherence of a compound to the surface material of the product. Preferably, the surface modification involves modification with one or more of the compounds. Surface modification, as defined herein in the context of the products, can be accomplished at any time and in any manner, including, for example, synthesis or production of the modified form of the product or material when the product or material is formed, addition of the chemical modification after the final product or material is formed, or at any time in between. Except where specifically and expressly provided to the contrary, the term “surface modification” refers to a structural property, regardless of how the structure was formed, and the structure is not limited to a structure made by any specific method. The surface can be any part of the product, such as side walls, top portion, and/or bottom portion.

In some embodiments, where explicitly indicated, addition or application of a material, compound, or composition to a starting material or intermediate before it is made into or incorporated into the final product can be specifically excluded. Thus, for example, chemical or surface modification of alginate or another polymer prior to the polymer being incorporated into a product, e.g. macrodevice, or other structure can be, in some embodiments, specifically excluded as the manner of producing a surface modification of the product or structure. As another example, coating a macrodevice, or other product with a material that was chemically modified prior to being applied as a coating can be, in some embodiments, specifically excluded as the manner of producing a surface modification of the macrodevice, or other product. However, for such embodiments where such specific exclusions are used, so long as the product was itself surface modified, coating of or addition to the product of another material that has chemical modifications does not alter the fact that the product was surface modified according to the meaning of the term used herein.

In some embodiments, the moieties or compounds modifying the product can be present on the surface or a surface of the product, and are not present, or are not present in a significant amount, elsewhere in the product, e.g., on internal or interior surfaces. In some embodiments, at least 50, 60, 70, 80, 90, 95, or 99% of the moieties or compounds are present on the surface or a surface of the product. In some embodiments, the moieties or compounds are present on the exterior face of the surface or a surface of the product, and are not present, or not present in a significant amount, elsewhere in the product, e.g., on internal or interior surfaces. In some embodiments, at least 50, 60, 70, 80, 90, 95, or 99% of the moieties or compounds are present on the external face of the surface or a surface of the product.

In some embodiments, the moieties or compounds modifying the product can be present on a portion or component of the product, and are not present, or are not present in a significant amount, elsewhere in the product. In some embodiments, at least 50, 60, 70, 80, 90, 95, or 99% of the moieties or compounds are present on the portion or component of the product. In some embodiments, the moieties or compounds are present on the exterior face of the portion or component of the product, and are not present, or not present in a significant amount, elsewhere in the product. In some embodiments, at least 50, 60, 70, 80, 90, 95, or 99% of the moieties or compounds are present on the external face of the portion or component of the product.

“Surface,” as used herein in the context of the products, refers to the exterior or outer boundary of a product. Generally, the surface or a surface of a product, e.g., macrodevice, corresponds to the idealized surface of a three dimensional solid that is topologically homeomorphic with the product. The surface or a surface of the product can be an exterior surface or an interior surface of the product. An exterior surface forms the outermost layer of a product or device. An interior surface surrounds an inner cavity of a product or device, such as the inner cavity of a tube. As an example, both the outside surface of a tube and the inside surface of a tube are part of the surface or a surface of the tube. However, internal surfaces of the product that are not in topological communication with the exterior surface, such as a tube with closed ends, can be excluded as the surface or a surface of a product. Preferred surfaces of a macrodevice to be chemically modified are the outside surface and surfaces that can contact immune system components. Where the product is porous or has holes in its mean (or idealized) surface, the internal faces of passages and holes would not be considered part of the surface or a surface of the product if its opening on the mean surface of the product is less than 5 nm. In some forms, when the surface contains a porous membrane, the surface of the porous membrane may or may not be chemically modified depending on the biocompatibility of the material used to make the porous membrane.

“Corresponding product” and “similar product,” as used herein, refers a product, e.g., macrodevice, that has, as far as is practical or possible, the same composition, structure, and construction as a reference product, e.g., macrodevice. The terms “corresponding” and “similar” can be used for the same meaning with any particular or subgroup of products or other materials described herein. For example, a “similar surface modification” refers a surface modification that has, as far as is practical or possible, the same composition, structure, and construction as a reference surface modification. “Control corresponding product” and “control similar product,” as used herein, refers a product, e.g., a macrodevice, that has, as far as is practical or possible, the same composition, structure, and construction as a reference product, e.g., reference macrodevice, except for one or more specified parameters. For example, a control corresponding product that lacks the chemical modification in reference to a chemically modified product refers to a product that has, as far as is practical or possible, the same composition, structure, and construction as a reference product except for the chemical modification. Generally, a product prior to chemical modification constitutes a control corresponding product to the chemically modified form of the product. The terms “control corresponding” and “control similar” can be used for the same meaning with any particular or subgroup of products or other materials described herein. For example, a “control similar surface modification” refers a surface modification that has, as far as is practical or possible, the same composition, structure, and construction as a reference surface modification except for one or more specified parameters. Components that are “control corresponding” or “control similar” relative to a reference component are useful as controls in assays assessing the effect of independent variables.

“Implanting,” as used herein, refers to the insertion or grafting into the body of a subject a product or material.

“Administering,” as used herein, refers to contacting a substance or product to the body of a subject. For example, administering a substance or a product includes contacting the skin of a subject and injecting or implanting a substance or product into the subject.

“Chemical compound,” as used herein, refers to an organic compound.

“High,” “higher,” “increases,” “elevates,” and “elevation,” as used herein, refer to increases above a reference level, e.g., a basal level, e.g., as compared to a control. “Low,” “lower,” “reduces,” and “reduction,” as used herein, refer to decreases below basal levels, e.g., as compared to a control.

“Improved,” as used herein, refers to a change that is desirable, which may be a higher or lower value of some measure.

“Subject,” as used herein, includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Treatment” and “treating,” as used herein, refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

“Beneficial effect,” as used herein, refers to any effect that is desired. In the context of the chemically modified products, beneficial effects include lower foreign body response, improved biocompatibility, and reduced immune response or reaction.

The phrase “effective amount,” as used herein in the context of a coating, generally refers to the amount of the coating applied to the implant in order to provide one or more clinically measurable endpoints, such as reduced foreign body response compared to an uncoated implant, an implant coated with an unmodified coating, or another suitable control. The phrase “effective amount,” as used herein in the context of a cell, capsule, product, device, material, composition, or compound, refers to a nontoxic but sufficient amount of the cell, capsule, product, device, material, composition, or compound to provide the desired result. The exact amount required may vary from subject to subject, depending on the species, age, and general condition of the subject; the severity of the disease that is being treated; the particular cell, capsule, product, device, material, composition, or compound used; its mode of administration; and other routine variables. An appropriate effective amount can be determined by one of ordinary skill in the art using only routine experimentation.

“Biological material” and “biomaterial,” as used herein, refers to any biological substance, including, but not limited to, tissue, cells, biological micromolecules, such as a nucleotides, amino acids, cofactors, and hormones, biological macromolecules, such as nucleic acids, polypeptides, proteins (for example enzymes, receptors, secretory proteins, structural and signaling proteins, hormones, ligands, etc.), polysaccharides, and/or any combination thereof.

“Cell,” as used herein, refers to individual cells, cell lines, primary cultures, or cultures derived from such cells unless specifically indicated.

“Culture,” as used herein, refers to a composition including cells, such as isolated cells, which can be of the same or a different type. “Cell line,” as used herein, refers to a permanently established cell culture that will proliferate indefinitely given appropriate fresh medium and space, thus making the cell line “immortal.” “Cell strain,” as used herein, refers to a cell culture having a plurality of cells adapted to culture, but with finite division potential. “Cell culture,” as used herein, is a population of cells grown on a medium such as agar.

Cells can be, for example, xenogeneic, autologous, or allogeneic. Cells can also be primary cells. Cells can also be cells derived from the culture and expansion of a cell obtained from a subject. For example, cells can also be stem cells or derived from stem cells. Cells can also be immortalized cells. Cells can also be genetically engineered to express or produce a protein, nucleic acid, or other product.

The terms “inhibit” and “reduce” means to reduce or decrease in activity or expression. This can be a complete inhibition or reduction of activity or expression, or a partial inhibition or reduction. Inhibition or reduction can be compared to a control or to a standard level. Inhibition can be 1, 10, 15, 20, 25, 30, 35, 40,45, 50, 55, 60, 65, 70, 75, 80, 85, 90, 95, 96, 97, 98, 99, or 100%.

“Small molecule” generally refers to an organic molecule that is less than about 2000 g/mol in molecular weight, less than about 1500 g/mol, less than about 1000 g/mol, less than about 800 g/mol, or less than about 500 g/mol. In some forms, small molecules are non-polymeric and/or non-oligomeric.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, arylalkyl, substituted arylalkyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, arylalkyl, substituted arylalkyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Except where specifically and expressly provided to the contrary, the term “substituted” refers to a structure, e.g., a chemical compound or a moiety on a larger chemical compound, regardless of how the structure was formed. The structure is not limited to a structure made by any specific method.

“Aryl,” as used herein, refers to C₅-C₂₆-membered aromatic, fused aromatic, fused heterocyclic, or biaromatic ring systems. Broadly defined, “aryl,” as used herein, includes 5-, 6-, 7-, 8-, 9-, 10-, 14-, 18-, and 24-membered single-ring aromatic groups, for example, benzene, naphthalene, anthracene, phenanthrene, chrysene, pyrene, corannulene, coronene, etc.

“Aryl” further encompasses polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF_{3,} —CCl₃), —CN, aryl, heteroaryl, and combinations thereof.

“Heterocycle,” “heterocyclic” and “heterocyclyl” are used interchangeably, and refer to a cyclic radical attached via a ring carbon or nitrogen atom of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Examples of heterocycles include, but are not limited to piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic, fused aromatic, biaromatic ring systems, or combinations thereof, in which one or more carbon atoms on one or more aromatic ring structures have been substituted with an heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics”. “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl”.

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH2-CF3-CCl₃), —CN, aryl, heteroaryl, and combinations thereof.

“Alkyl,” as used herein, refers to the radical of saturated aliphatic groups, including straight-chain alkyl, alkenyl, or alkynyl groups, branched-chain alkyl, cycloalkyl (alicyclic), alkyl substituted cycloalkyl groups, and cycloalkyl substituted alkyl. In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfoxide, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

“Alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NRR′, wherein R and R′ are independently hydrogen, alkyl, or aryl, and wherein the nitrogen atom is optionally quaternized; —SR, wherein R is hydrogen, alkyl, or aryl; —CN; —NO_{2;}—COOH; carboxylate; —COR, —COOR, or —CON(R)_{2,} wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, haloalkyl (such as —CF₃, —CH₂—CF_{3,} —CCl₃); —CN; —NCOCOCH₂CH_{2,} —NCOCOCHCH; —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl, sulfoxide, and sulfonate), and silyl groups, as well as ethers, alkylthios, carbonyls (including ketones, aldehydes, carboxylates, and esters), haloalkyls, —CN and the like. Cycloalkyls can be substituted in the same manner.

The terms “alkenyl” and “alkynyl” refer to unsaturated aliphatic groups analogous in length and possible substitution to the alkyls described above, but that contain at least one double or triple bond, respectively.

The term “substituted alkenyl” refers to alkenyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “substituted alkynyl” refers to alkynyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenyl” is art recognized, and refers to the aromatic moiety —C₆H_{5,} i.e., a benzene ring without one hydrogen atom.

The term “substituted phenyl” refers to a phenyl group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Amino” and “Amine,” as used herein, are art-recognized and refer to both substituted and unsubstituted amines, e.g., a moiety that can be represented by the general formula:

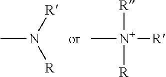

wherein, R, R′, and R″ each independently represent a hydrogen, substituted or unsubstituted alkyl, substituted or unsubstituted alkenyl, substituted or unsubstituted alkynyl, substituted or unsubstituted carbonyl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ (and optionally R″) each independently represent a hydrogen atom, substituted or unsubstituted alkyl, a substituted or unsubstituted alkenyl, or —(CH₂)ₘ—R′″. Thus, the term ‘alkylamine’ as used herein refers to an amine group, as defined above, having a substituted or unsubstituted alkyl attached thereto (i.e. at least one of R, R′, or R″ is an alkyl group).

“Carbonyl,” as used herein, is art-recognized and includes such moieties as can be represented by the general formula:

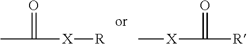

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defines as above, the moiety is also referred to as a carboxyl group. When X is oxygen and R is hydrogen, the formula represents a ‘carboxylic acid’. Where X is oxygen and R′ is hydrogen, the formula represents a ‘formate’. Where X is oxygen and R or R′ is not hydrogen, the formula represents an “ester”. In general, where the oxygen atom of the above formula is replaced by a sulfur atom, the formula represents a ‘thiocarbonyl’ group. Where X is sulfur and R or R′ is not hydrogen, the formula represents a ‘thioester.’ Where X is sulfur and R is hydrogen, the formula represents a ‘thiocarboxylic acid.’ Where X is sulfur and R′ is hydrogen, the formula represents a ‘thioformate.’ Where X is a bond and R is not hydrogen, the above formula represents a ‘ketone.’ Where X is a bond and R is hydrogen, the above formula represents an ‘aldehyde.’

The term “substituted carbonyl” refers to a carbonyl, as defined above, wherein one or more hydrogen atoms in R, R′ or a group to which the moiety

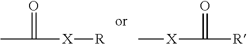

is attached, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “carboxyl” is as defined above for the formula

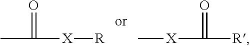

and is defined more specifically by the formula —R^{iv}COOH, wherein R^{iv} is an alkyl, alkenyl, alkynyl, cycloalkyl, heterocyclyl, alkylaryl, arylalkyl, aryl, or heteroaryl. In preferred embodiments, a straight chain or branched chain alkyl, alkenyl, and alkynyl have 30 or fewer carbon atoms in its backbone (e.g., C₁-C₃₀ for straight chain alkyl, C₃-C₃₀ for branched chain alkyl, C₂-C₃₀ for straight chain alkenyl and alkynyl, C₃-C₃₀ for branched chain alkenyl and alkynyl), preferably 20 or fewer, more preferably 15 or fewer, most preferably 10 or fewer Likewise, preferred cycloalkyls, heterocyclyls, aryls and heteroaryls have from 3-10 carbon atoms in their ring structure, and more preferably have 5, 6 or 7 carbons in the ring structure.

The term “substituted carboxyl” refers to a carboxyl, as defined above, wherein one or more hydrogen atoms in R^{iv} are substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Heteroalkyl,” as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

The terms “alkoxyl” or “alkoxy,” “aroxy” or “aryloxy,” generally describe compounds represented by the formula —OR^{v}, wherein R^{v} includes, but is not limited to, substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. The term alkoxy also includes cycloalkyl, heterocyclyl, cycloalkenyl, heterocycloalkenyl, and arylalkyl having an oxygen radical attached to at least one of the carbon atoms, as valency permits.

The term “substituted alkoxy” refers to an alkoxy group having one or more substituents replacing one or more hydrogen atoms on one or more carbons of the alkoxy backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenoxy” is art recognized, and refers to a compound of the formula —OR^{v} wherein R^{v} is (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such sub stituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto.

The term “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenylthio” is art recognized, and refers to —S—C₆H_{5,} i.e., a phenyl group attached to a sulfur atom.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. “Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Arylalkyl,” as used herein, refers to an alkyl group that is substituted with a substituted or unsubstituted aryl or heteroaryl group.

“Alkylaryl,” as used herein, refers to an aryl group (e.g., an aromatic or hetero aromatic group), substituted with a substituted or unsubstituted alkyl group.

The terms “amide” or “amido” are used interchangeably, refer to both “unsubstituted amido” and “substituted amido” and are represented by the general formula:

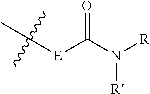

wherein, E is absent, or E is substituted or unsubstituted alkyl, substituted or unsubstituted alkenyl, substituted or unsubstituted alkynyl, substituted or unsubstituted aralkyl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ each independently represent a hydrogen atom, substituted or unsubstituted alkyl, a substituted or unsubstituted alkenyl, or —(CH₂)ₘ—R′″. When E is oxygen, a carbamate is formed. The carbamate cannot be attached to another chemical species, such as to form an oxygen-oxygen bond, or other unstable bonds, as understood by one of ordinary skill in the art.

The term “sulfonyl” is represented by the formula

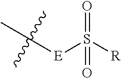

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ−R′″, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of E and R can be substituted or unsubstituted amine, to form a “sulfonamide” or “sulfonamido.” The substituted or unsubstituted amine is as defined above.

The term “substituted sulfonyl” represents a sulfonyl in which E, R, or both, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

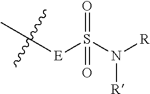

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide.

The term “sulfoxide” is represented by the formula

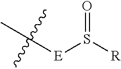

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “phosphonyl” is represented by the formula

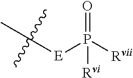

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein, independently of E, R^{vi} and R^{vii} are independently hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the P atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “substituted phosphonyl” represents a phosphonyl in which E, R^{vi} and R^{vii} are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phosphoryl” defines a phoshonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “polyaryl” refers to a chemical moiety that includes two or more aryls, heteroaryls, and combinations thereof. The aryls, heteroaryls, and combinations thereof, are fused, or linked via a single bond, ether, ester, carbonyl, amide, sulfonyl, sulfonamide, alkyl, azo, and combinations thereof.

The term “substituted polyaryl” refers to a polyaryl in which one or more of the aryls, heteroaryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “C₃-C₂₀ cyclic” refers to a substituted or unsubstituted cycloalkyl, substituted or unsubstituted cycloalkenyl, substituted or unsubstituted cycloalkynyl, substituted or unsubstituted heterocyclyl that have from three to 20 carbon atoms, as geometric constraints permit. The cyclic structures are formed from single or fused ring systems. The substituted cycloalkyls, cycloalkenyls, cycloalkynyls and heterocyclyls are substituted as defined above for the alkyls, alkenyls, alkynyls and heterocyclyls, respectively.

The terms “hydroxyl” and “hydroxy” are used interchangeably and are represented by —OH.

The terms “thiol” and “sulfhydryl” are used interchangeably and are represented by —SH.

The term “oxo” refers to ═O bonded to a carbon atom.

The terms “cyano” and “nitrile” are used interchangeably to refer to —CN.

The term “nitro” refers to —NO₂.

The term “phosphate” refers to —O—PO_{3.}

The term “azide” or “azido” are used interchangeably to refer to —N_{3.}

The term “substituted C₁-Cₓ alkyl” refers to alkyl groups having from one to x carbon atoms, wherein at least one carbon atom is substituted, wherein “x” is an integer from one to ten. The term “unsubstituted C₁-Cₓ alkyl” refers to alkyl groups having from one to x carbon atoms that are not substituted, wherein “x” is an integer from one to ten.

The term “substituted C₁-Cₓ alkylene” refers to alkylene groups having from one to x carbon atoms, wherein at least one carbon atom is substituted, wherein “x” is an integer from one to ten. The term “unsubstituted C₁-Cₓ alkylene” refers to alkylene groups having from one to x carbon atoms that are not substituted, wherein “x” is an integer from one to ten. The term “alkylene” as used herein, refers to a moiety with the formula —(CH₂)ₐ—, wherein “a” is an integer from one to ten.

The term “substituted C₂-Cₓ alkenyl” refers to alkenyl groups having from two to x carbon atoms, wherein at least one carbon atom is substituted, wherein “x” is an integer from two to ten. The term “unsubstituted C₂-Cₓ alkenyl” refers to alkenyl groups having from two to x carbon atoms that are not substituted, wherein “x” is an integer from two to ten.

The term “substituted C₂-Cₓ alkynyl” refers to alkynyl groups having from two to x carbon atoms, wherein at least one carbon atom is substituted, wherein “x” is an integer from two to ten. The term “unsubstituted C₂-Cₓ alkynyl” refers to alkynyl groups having from two to x carbon atoms that are not substituted, wherein “x” is an integer from two to ten.

The term “substituted C₁-Cₓ alkoxy” refers to alkoxy groups having from one to x carbon atoms, wherein at least one carbon atom is substituted, wherein “x” is an integer from one to ten. The term “unsubstituted C₁-Cₓ alkoxy” refers to alkoxy groups having from one to x carbon atoms that are not substituted, wherein “x” is an integer from one to ten.

The term “substituted C₁-Cₓ alkylamino” refers to alkylamino groups having from one to x carbon atoms, wherein at least one carbon atom is substituted, wherein “x” is an integer from one to ten. The term “unsubstituted C₁-Cₓ alkylamino” refers to alkyl groups having from one to x carbon atoms that are not substituted, wherein “x” is an integer from one to ten. The terms “alkylamine” and “alkylamino” are used interchangeably. In any alkylamino, where the nitrogen atom is substituted with one, two, or three substituents, the nitrogen atom can be referred to as a secondary, tertiary, or quartenary nitrogen atom, respectively.

The term “substituted C₁-Cₓ alkylthio” refers to alkylthio groups having from one to x carbon atoms, wherein at least one carbon atom is substituted, wherein “x” is an integer from one to ten. The term “unsubstituted C₁-Cₓ alkylthio” refers to alkylthio groups having from one to x carbon atoms that are not substituted, wherein “x” is an integer from one to ten.

The term “substituted C₁-Cₓ carbonyl” refers to carbonyl groups having from one to x carbon atoms, wherein at least one carbon atom is substituted, wherein “x” is an integer from one to ten. The term “unsubstituted C₁-Cₓ carbonyl” refers to carbonyl groups having from one to x carbon atoms that are not substituted, wherein “x” is an integer from one to ten.

The term “substituted C₁-Cₓ carboxyl” refers to carboxyl groups having from one to x carbon atoms, wherein at least one carbon atom is substituted, wherein “x” is an integer from one to ten. The term “unsubstituted C₁-Cₓ carboxyl” refers to carboxyl groups having from one to x carbon atoms that are not substituted, wherein “x” is an integer from one to ten.

The term “substituted C₁-Cₓ amido” refers to amido groups having from one to x carbon atoms, wherein at least one carbon atom is substituted, wherein “x” is an integer from one to ten. The term “unsubstituted C₁-Cₓ amido” refers to amido groups having from one to x carbon atoms that are not substituted, wherein “x” is an integer from one to ten.

The term “substituted C₁-Cₓ sulfonyl” refers to sulfonyl groups having from one to x carbon atoms, wherein at least one carbon atom is substituted, wherein “x” is an integer from one to ten. The term “unsubstituted C₁-Cₓ sulfonyl” refers to sulfonyl groups having from one to x carbon atoms that are not substituted, wherein “x” is an integer from one to ten.

The term “substituted C₁-Cₓ sulfonic acid” refers to sulfonic acid groups having from one to x carbon atoms, wherein at least one carbon atom is substituted, wherein “x” is an integer from one to ten. The term “unsubstituted C₁-Cₓ sulfonic acid” refers to sulfonic acid groups having from one to x carbon atoms that are not substituted, wherein “x” is an integer from one to ten.

The term “substituted C₁-Cₓ sulfamoyl” refers to sulfamoyl groups having from one to x carbon atoms, wherein at least one carbon atom is substituted, wherein “x” is an integer from one to ten. The term “unsubstituted C₁-Cₓ sulfamoyl” refers to sulfamoyl groups having from one to x carbon atoms that are not substituted, wherein “x” is an integer from one to ten.

The term “substituted C₁-Cₓ sulfoxide” refers to sulfoxide groups having from one to x carbon atoms, wherein at least one carbon atom is substituted, wherein “x” is an integer from one to ten. The term “unsubstituted C₁-Cₓ sulfoxide” refers to sulfoxide groups having from one to x carbon atoms that are not substituted, wherein “x” is an integer from one to ten.

The term “substituted C₁-Cₓ phosphoryl” refers to phosphoryl groups having from one to x carbon atoms, wherein at least one carbon atom is substituted, wherein “x” is an integer from one to ten. The term “unsubstituted C₁-Cₓ phosphoryl” refers to phosphoryl groups having from one to x carbon atoms that are not substituted, wherein “x” is an integer from one to ten.

The term “substituted C₁-Cₓ phosphonyl” refers to phosphonyl groups having from one to x carbon atoms, wherein at least one carbon atom is substituted, wherein “x” is an integer from one to ten. The term “unsubstituted C₁-Cₓ phosphonyl” refers to phosphonyl groups having from one to x carbon atoms that are not substituted, wherein “x” is an integer from one to ten.

The term “substituted C₀-Cₓ sulfonyl” refers to sulfonyl groups having from zero to x carbon atoms, wherein, if present, at least one carbon atom is substituted, wherein “x” is an integer from zero to ten. The term “unsubstituted C₀-Cₓ sulfonyl” refers to sulfonyl groups having from zero to x carbon atoms that are not substituted, wherein “x” is an integer from zero to ten.

The term “substituted C₀-Cₓ sulfonic acid” refers to sulfonic acid groups having from zero to x carbon atoms, wherein, if present, at least one carbon atom is substituted, wherein “x” is an integer from zero to ten. The term “unsubstituted C₀-Cₓ sulfonic acid” refers to sulfonic acid groups having from zero to x carbon atoms that are not substituted, wherein “x” is an integer from zero to ten.

The term “substituted C₀-Cₓ sulfamoyl” refers to sulfamoyl groups having from zero to x carbon atoms, wherein, if present, at least one carbon atom is substituted, wherein “x” is an integer from zero to ten. The term “unsubstituted C₀-Cₓ sulfamoyl” refers to sulfamoyl groups having from zero to x carbon atoms that are not substituted, wherein “x” is an integer from zero to ten.

The term “substituted C₀-Cₓ sulfoxide” refers to sulfoxide groups having from zero to x carbon atoms, wherein at least one carbon atom is substituted, wherein “x” is an integer from zero to ten. The term “unsubstituted C₀-Cₓ sulfoxide” refers to sulfoxide groups having from zero to x carbon atoms that are not substituted, wherein “x” is an integer from zero to ten.

The term “substituted C₀-Cₓ phosphoryl” refers to phosphoryl groups having from zero to x carbon atoms, wherein, if present, at least one carbon atom is substituted, wherein “x” is an integer from zero to ten. The term “unsubstituted C₀-Cₓ phosphoryl” refers to phosphoryl groups having from zero to x carbon atoms that are not substituted, wherein “x” is an integer from zero to ten.

The term “substituted C₀-Cₓ phosphonyl” refers to phosphonyl groups having from zero to x carbon atoms, wherein, if present, at least one carbon atom is substituted, wherein “x” is an integer from zero to ten. The term “unsubstituted C₀-Cₓ phosphonyl” refers to phosphonyl groups having from zero to x carbon atoms that are not substituted, wherein “x” is an integer from zero to ten.

The terms substituted “Cₓ alkyl,” “Cₓ alkylene,” “Cₓ alkenyl,” “Cₓ alkynyl,” “Cₓ alkoxy,” “Cₓ alkylamino,” “Cₓ alkylthio,” “Cₓ carbonyl,” “Cₓ carboxyl,” “Cₓ amido,” “Cₓ sulfonyl,” “Cₓ sulfonic acid,” “Cₓ sulfamoyl,” “Cₓ phosphoryl,” and “Cₓ phosphonyl” refer to alkyl, alkylene, alkenyl, alkynyl, alkoxy, alkylamino, alkylthio, carbonyl, carboxyl, amido, sulfonyl, sulfonic acid, sulfamoyl, sulfoxide, phosphoryl, and phosphonyl groups, respectively, having x carbon atoms, wherein at least one carbon atom is substituted, wherein “x” is an integer from one to ten. The terms unsubstituted “Cₓ alkyl,” “Cₓ alkylene,” “Cₓ alkenyl,” “Cₓ alkynyl,” “Cₓ alkoxy,” “Cₓ alkylamino” , “Cₓ alkylthio,” “Cₓ carbonyl,” “Cₓ carboxyl,” “Cₓ amido,” “Cₓ sulfonyl,” “Cₓ sulfonic acid,” “Cₓ sulfamoyl,” “Cₓ phosphoryl,” and “Cₓ phosphonyl” refer to alkyl, alkylene, alkenyl, alkynyl, alkoxy, alkylamino, alkylthio, carbonyl, carboxyl, amido, sulfonyl, sulfonic acid, sulfamoyl, sulfoxide, phosphoryl, and phosphonyl groups, respectively, having x carbon atoms that are not substituted, wherein “x” is an integer from one to ten.

The terms unsubstituted “C₀ sulfonyl,” “C₀ sulfonic acid,” “C₀ sulfamoyl,” “C₀ phosphoryl,” and “C₀ phosphonyl” refer to alkyl, alkylene, alkenyl, alkynyl, alkoxy, alkylamino, alkylthio, carbonyl, carboxyl, amido, sulfonyl, sulfonic acid, sulfamoyl, sulfoxide, phosphoryl, and phosphonyl groups, respectively, having zero carbon atoms that are not substituted.

### II. Composition

Described herein, are macrodevices containing a body (or micro-fabricated body) having at least one or multiple compartments and a porous membrane. Each of the one or multiple compartments contain one or more side walls, a top portion preferably sealed by the porous membrane via attachment to an outline of the one or more side walls, and a bottom portion. Preferably, the body of the macrodevice is formed via one or more micro-fabrication techniques. Preferably, micro-fabricated body and/or porous membrane contain a material selected from polydimethoxysiloxane, medical grade silicone, polycarbonate, polyurethane, or a combination thereof.

The macrodevices described herein, have the advantage that one or multiple compartments can be used to spatially localize substances at a desired distance from the porous membrane, for efficient diffusion of materials into the compartments. In particular, the micro-fabricated body provides precise control over the position of cells relative to the membrane, allowing for sufficient amounts of oxygen and nutrients to reach cells, while also providing easy diffusion of secreted therapeutic agents from the compartments. Further, the body can be designed to control the spatial location of cells within macrodevice, such that two or more cells can be placed in close proximity, while remaining physically separated. In some forms, the body contains integrated fluidic channels that serve as conduits for accessing the compartments. In general, the macrodevices can have any shape in which at least two dimensions are not the same. In some forms, the macrodevices have an oblong shape, rounded corners, or both, and a thickness that is selected to enhance the diffusion of nutrients into and out of a compartment, in order to provide long-term functional viability of the encapsulated substances (e.g. cells). These properties are not possible in current implantation devices.

The size of the macrodevice can be scalable based on the desired application, such as site of implantation. For example, the macrodevice can have an overall length, X, an overall width, Y, and an overall height, Z, wherein each X, Y, and Z is independently an integer between 10 μm and 50 mm, inclusive, with the proviso that X and Y are selected such that X is always greater than Y. In some forms, Z is the perpendicular distance from the porous membrane to a bottom portion, such as the bottom of a compartment. As an example, the perpendicular distance can be between 10 μm and 1 mm, such as about 0.25 mm.

In some forms, the macrodevice contains multiple compartments arranged in a one-dimensional array, two-dimensional array, or three-dimensional array. When present in a one-dimensional array, the compartments are adjacent to each other in succession, only as a single row, or stacked on each other only as a single column. When present in a two-dimensional array, the compartments are arranged in at least two rows or columns, wherein successive rows or columns are adjacent to each other. In a three-dimensional array, at least one row is stacked on a two-dimensional array of compartments.

In some forms, the macrodevices encapsulate one or more substances, such as cells, in the one or more compartments and provide immunoprotection to the encapsulated substances by preventing infiltration of the macrodevice by the immune cells of a subject following implantation of the device. Immunoprotection of the macrodevice can depend on factors such as the sizes of the pores in the porous membrane, the presence of polymers and/or small molecules that are used to chemically modify a surface of the macrodevice, or a combination of these factors. The porous membrane has a thickness and/or pore size that allows diffusion of therapeutic agents, diagnostic agents, prophylactic agents, nutrients, oxygen, or combinations thereof, but blocks host immune cells from entering the compartments. The size of the pores can be between 0.1 μm and 3.0 μm, inclusive, preferably between 0.1 μm and 0.8 μm, inclusive. The porous membrane can also have a thickness between 0.5 μm and 100 μm, inclusive, 0.5 μm and 75 μm, inclusive, 0.5 μm and 50 μm, inclusive, 0.5 μm and 25 μm, inclusive, 1 μm and 20 μm, inclusive. Exemplary thickness values can include 0.5 μm, 1 μm, 2 μm, 3 μm, 4 μm, 5 μm, 10 μm, 15 μm, and 20 μm, etc. The polymers and/or small molecules can completely or partially cover the surface of the macrodevice. These chemical modifications can also impart properties such as reduced fibrosis of the macrodevices, thereby allowing in vivo delivery of therapeutic agents for extended periods of time. These polymers and/or small molecules may also be referred to as a biocompatible component,

A. Macrodevices

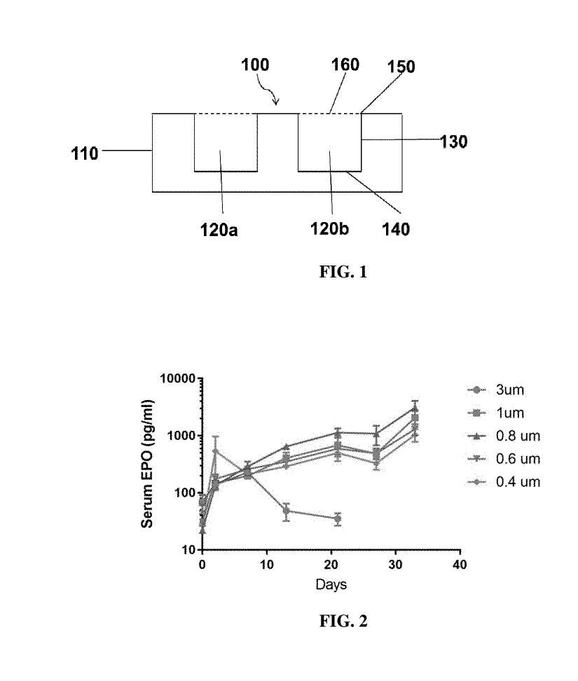

FIG. 1 is a schematic of a macrodevice 100. The macrodevice 100 contains a body 110 that includes one or multiple compartments 120a and/or 120b. A compartment (e.g. 120a) contains one or more side walls 130, a top portion 150 containing a porous membrane 160. The outlines of the top portion 150 and the bottom portion 140 of a compartment 120a can be the same or different. In some forms, the body 110 has a largest dimension, such that the macrodevice 100 is suitable for implantation into a subject. Preferably, the body is fabricated using one or more micro-fabrication techniques. The membrane can be attached to the body before or after cells are added to a compartment of the macrodevice. The porous membrane can be attached to the body via heat pressing, laser welding, chemical bonding, glues, or combinations thereof.

B. Small Molecules

The small molecules coating a surface of the macrodevice can have the structure:

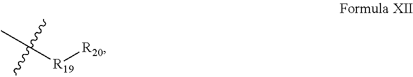

In some forms, R₁₉ and R₂₀ are independently unsubstituted alkyl, substituted alkyl, unsubstituted alkenyl, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, —O—, —S—, —NH—NHC(O)—, —N═N—, —N═CH—, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group.

In some forms, R₁₉ is —C(O)NH—, —C(O)O—, —NHC(O)—, —OC(O)—, —O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

In some forms of Formula XII, R₂₀ has the structure:

-Az-Bz-(—Cz)δ, Formula XIII

wherein δ is an integer between 0 and 10, inclusive, preferably δ is 1.

In some forms of Formula VII, Az can be:

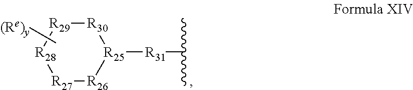

wherein R₃₁ in Az is —(CR₃₂R₃₂)ₚ—; p is an integer from 0 to 5; each R₃₂ is hydrogen, unsubstituted alkyl, or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R_{25,} R_{26,} R_{27,} R_{28,} R_{29,} and R₃₀ are independently C or N, wherein the bonds between adjacent R₂₅ to R₃₀ are double or single according to valency, and wherein R₂₅ to R₃₀ are bound to none, one, or two hydrogens according to valency.

In some forms of Formula XIV, each R₃₂ is hydrogen, and p is 1.

In some forms of Formula XIV, each R₃₂ is hydrogen, p is 1, R₂₅ is C, and R₂₆-R₃₀ are CH, and the bonds between R₂₅ and R_{26,} between R₂₇ and R₂₆, and between R₂₉ and R₃₀ are double bonds.

In some forms of Formula XIV, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, and y is 1.

In some forms of Formula XIV, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, y is 1, and R^{e is Bz.}

In some forms of Formula XIV, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, y is 1, and R^{e} contains a substituted heteroaryl group.

In some forms of Formula XIV, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, y is 1, R^{e} contains a substituted heteroaryl group, wherein the substituted heteroaryl group is a substituted triazole.

In some forms of Formula XIII, Az can be:

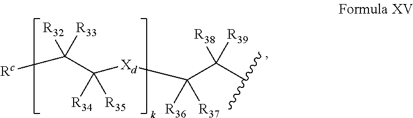

wherein R_{32,} R_{33,} R_{34,} R_{35,} R_{36,} R_{37,} R_{38,} and R₃₉ in Az are independently hydrogen, unsubstituted alkyl, substituted alkyl, unsubstituted alkenyl, substituted alkenyl, unsubstituted alkynyl, substituted alkynyl, unsubstituted phenyl, substituted phenyl, unsubstituted aryl, substituted aryl, unsubstituted heteroaryl, substituted heteroaryl, unsubstituted arylalkyl, substituted arylalkyl, unsubstituted alkoxy, substituted alkoxy, unsubstituted aroxy, substituted aroxy, unsubstituted carbonyl, substituted carbonyl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, substituted C₃-C₂o heterocyclic, poly(ethylene glycol), or poly(lactic-co-glycolic acid); k is an integer from 0 to 20; each X_{d} is independently absent, O, or S; and R^{c} can be Bz.

In some forms of Formula XV, Xd is O. In some forms of Formula IX, Xd is O, and R₃₂-R₃₉ are hydrogen.

In some forms of Formula XV, Xd is O, R₃₂-R₃₉ are hydrogen, and k is an integer between 1 and 5, inclusive, preferably 3.

In some forms of Formula XIII or IX, Bz can be:

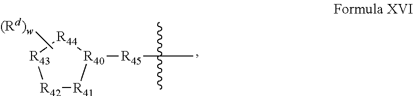

wherein R₄₅ in Bz is —(CR₄₆R₄₆)ₚ—; p is an integer from 0 to 5; each R₄₆ is hydrogen, unsubstituted alkyl, or substituted alkyl; each R^{d} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; w is an integer between 0 and 4, inclusive; each R_{40,} R_{41,} R_{42,} R_{43,} and R_{44,} are independently C or N, wherein the bonds between adjacent R₄₀ to R₄₄ are double or single according to valency, and wherein R₄₀ to R₄₄ are bound to none, one, or two hydrogens according to valency.

In some forms of Formula XVI, p is 0.

In some forms of Formula XVI, p is 0, and R₄₀-R₄₂ are N.

In some forms of Formula XVI, p is 0, R₄₀-R₄₂ are N, and R₄₃ and R₄₄ are C.

In some forms, Formula XVI is:

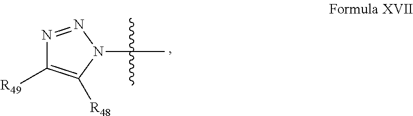

wherein R₄₈ and R₄₉ are independently hydrogen,

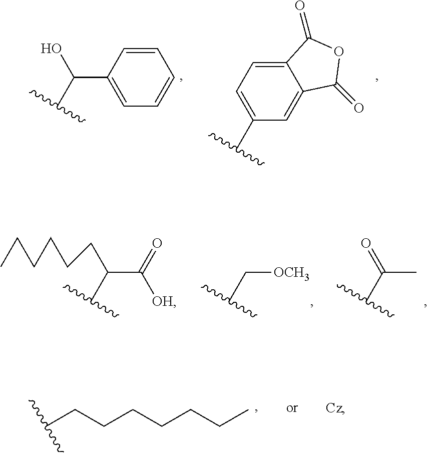

with the proviso that at least one of R₄₈ and R₄₉ is not hydrogen.

In some forms of Formula XIII or Formula XVII, Cz can be:

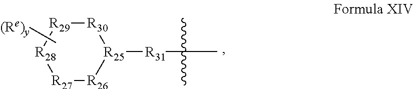

wherein R₃₁ in Cz is —(CR₃₂R₃₂)ₚ— or —(CR₃₂R₃₂)ₚ—X_{b}—(CR₃₂R₃₂)_{q}—; p and q are independently integers between 0 to 5, inclusive; each R₃₂ is hydrogen, unsubstituted alkyl, or substituted alkyl; X_{b} is absent, —O—, —S—, —S(O)—, —S(O)₂—, or NR_{47;} R₄₇ is unsubstituted alkyl or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R_{25,} R_{26,} R_{27,} R₂₈, R_{29,} and R₃₀ are independently C or N, wherein the bonds between adjacent R₂₅ to R₃₀ are double or single according to valency, and wherein R₂₅ to R₃₀ are bound to none, one, or two hydrogens according to valency.

In some forms of Formula XIV, R₃₁ is —(CR₃₂R₃₂)ₚ—, each R₃₂ is hydrogen, and p is 1.

In some forms of Formula XIV, R₃₁ is —(CR₃₂R₃₂)ₚ—, each R₃₂ is hydrogen, p is 1, and R₂₅ is N.

In some forms of Formula XIV, R₃₁ is —(CR₃₂R₃₂)ₚ—, each R₃₂ is hydrogen, p is 1, R₂₅ is N, and R₂₈ is S(O)₂.

In some forms of Formula XIV, R₃₁ is —(CR₃₂R₃₂)ₚ—, each R₃₂ is hydrogen, p is 1, R₂₅ is N, R₂₈ is S(O)_{2,} and R_{26,} R_{27,} R_{29,} and R₃₀ are CH₂.

In some forms of Formula XIV, R₃₁ is —(CR₃₂R₃₂)ₚ—, each R₃₂ is hydrogen, p is 1, R₂₅ is N, R₂₈ is S(O)_{2,} R_{26,} R_{27,} R_{29,} and R₃₀ are CH_{2,} and y is 0, i.e.,

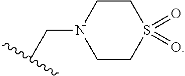 [SMILES: *CN1CCS(=O)(=O)CC1]

In some forms of Formula XIV, R₃₁ is —(CR₃₂R₃₂)ₚ—X_{b}—(CR₃₂R₃₂)_{q}—, each R₃₂ is hydrogen, and p is 0.

In some forms of Formula XIV, R₃₁ is —(CR₃₂R₃₂)ₚ—X_{b}—(CR₃₂R₃₂)_{q}—, each R₃₂ is hydrogen, p is 0, and q is 1.

In some forms of Formula XIV, R₃₁ is —(CR₃₂R₃₂)ₚ—X_{b}—(CR₃₂R₃₂)_{q}—, each R₃₂ is hydrogen, p is 0, q is 1, and X_{b} is O or —S(O)₂—.

In some forms of Formula XIV, R₃₁ is —(CR₃₂R₃₂)ₚ—X_{b}—(CR₃₂R₃₂)_{q}—, each R₃₂ is hydrogen, p is 0, q is 1, X_{b} is O, and R₂₆ is O.

In some forms of Formula XIV, R₃₁ is —(CR₃₂R₃₂)ₚ—X_{b}—(CR₃₂R₃₂)_{q}—, each R₃₂ is hydrogen, p is 0, q is 1, X_{b} is O, R₂₆ is O, and R₂₅ is CH.

In some forms of Formula XIV, R₃₁ is —(CR₃₂R₃₂)ₚ—X_{b}—(CR₃₂R₃₂)_{q}—, each R₃₂ is hydrogen, p is 0, q is 1, X_{b} is O, R₂₆ is O, R₂₅ is CH, R₂₇-R₃₀ are CH_{2,} and y is 0, i.e.,

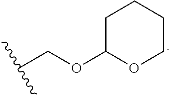 [SMILES: *COC1CCCCO1]

In some forms of Formula XIV, R₃₁ is —(CR₃₂R₃₂)ₚ—X_{b}—(CR₃₂R₃₂)_{q}—, each R₃₂ is hydrogen, p is 0, q is 1, X_{b} is —S(O)₂—, R₂₅ is C, R₂₆-R₃₀ are CH, and the bonds between R₂₅ and R_{26,} between R₂₇ and R_{28,} and between R₂₉ and R₃₀ are double bonds, i.e.,

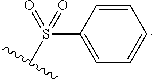 [SMILES: *S(=O)(=O)c1ccccc1]

In some forms of Formula XIV, R₃₁ is —(CR₃₂R₃₂)ₚ—, and p is 0.

In some forms of Formula XIV, R₃₁ is —(CR₃₂R₃₂)ₚ—, p is 0, R₂₅ is C, and R₂₆-R₃₀ are CH, and the bonds between R₂₅ and R_{26,} between R₂₇ and R_{28,} and between R₂₉ and R₃₀ are double bonds.

In some forms of Formula XIV, R₃₁ is —(CR₃₂R₃₂)ₚ—, p is 0, R₂₅ is C, and R₂₆-R₃₀ are CH, the bonds between R₂₅ and R_{26,} between R₂₇ and R_{28,} and between R₂₉ and R₃₀ are double bonds, and y is 0 or 1.

In some forms of Formula XIV, R₃₁ is —(CR₃₂R₃₂)ₚ—, p is 0, R₂₅ is C, and R₂₆-R₃₀ are CH, the bonds between R₂₅ and R_{26,} between R₂₇ and R_{28,} and between R₂₉ and R₃₀ are double bonds, y is 1, and R^{e} is —NH₂, —OCH₃, or —CH₂OH, i.e.,

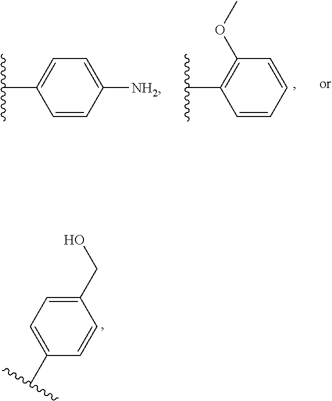

respectively.

In some forms of Formula XIV, R₃₁ is —(CR₃₂R₃₂)ₚ—, p is 0, R₂₅ is C, R₂₇ is N, R_{26,} R₂₈-R₃₀ are CH, the bonds between R₂₅ and R_{26,} between R₂₇ and R_{28,} and between R₂₉ and R₃₀ are double bonds, and y is 0, i.e.,

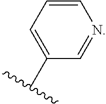 [SMILES: *c1cccnc1]

In some forms of Formula XIV, R₃₁ is —(CR₃₂R₃₂)ₚ—, p is 0, R₂₅ is C(OH), and R₂₆-R₃₀ are CH_{2,} and y is 0, i.e.,

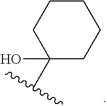 [SMILES: *C1(O)CCCCC1]

In some forms, the small molecule contains the moiety:

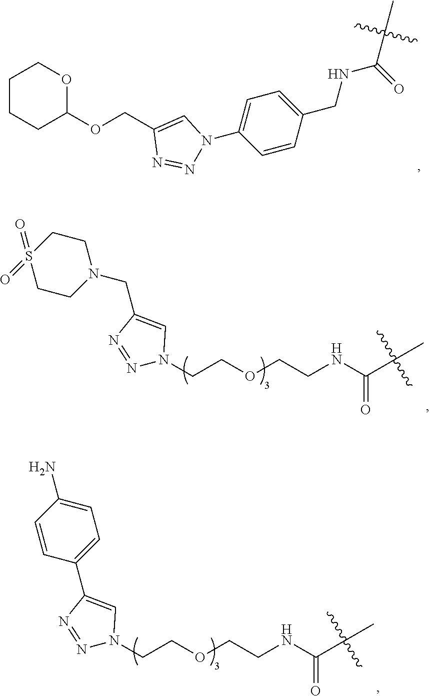

or combinations thereof.

C Polymers

1. Zwitterionic Polymers

The polymers used to coat a surface of the macrodevice contain a backbone and a plurality of side chains formed by monomer subunit A, and optionally monomer subunits B, C, or both. Each A within the polymer is a zwitterionic monomer. The A subunits can be formed from monomers having the same zwitterion or from monomers having different zwitterions. Each B is independently a monomer with a reactive side chain. The B subunits can be formed from monomers having the same reactive side chain or from monomers having different reactive side chains. Each C is independently a hydrophobic monomer or a neutral hydrophilic monomer. The C subunits can be formed from the monomers with having the same hydrophobic or neutral side chain or from monomers having different hydrophobic or neutral side chains.

In some forms, the zwitterionic polymers can be mixed or blended with other non-zwitterionic polymers to form a mixture. The non-zwitterionic polymers can be hydrophilic, hydrophobic, or amphiphilic.

The zwitterionic polymers can be biocompatible, biodegradable, non-biodegradable, or a combination thereof. The polymers can be purified after synthesis to remove any unreacted or partially reacted contaminants present with the chemically polymeric product.

2. Polymer Backbone

The polymer backbone can be neutral (e.g., polyalkylene or polyether) or contain permanently charged moieties (e.g., cyclic or acyclic quaternized nitrogen atoms), or even zwitterionic backbones (e.g., phosphorylcholine backbones). Therefore, the backbone of the polymers can be formed from polymers that include, but are not limited to, poly(acrylate), poly(methacrylate), poly(acrylamide), poly(methacrylamide), poly(vinyl alcohol), poly(ethylene vinyl acetate), poly(vinyl acetate), polyolefin, polyester, polyanhydride, poly (orthoester), polyamide, polyamine, polyether, polyazine, poly(carbonate), polyetheretherketone (PEEK), polyguanidine, polyimide, polyketal, poly(ketone), polyphosphazine, polysaccharide, polysiloxane, polysulfone, polyurea, polyurethane, combinations thereof.

3. Monomers used to Form the Polymers

i. Zwitterionic Monomers

Each zwitterionic monomer within the polymer is denoted A. The zwitterionic monomers contain carboxybetaine moieties, sulfobetaine moieties, and phosphoryl choline moieties.

The zwitterionic moieties can be represented by:

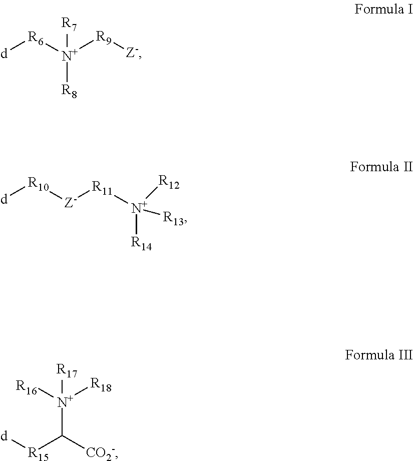

wherein d is the point of covalent attachment of the zwitterion to the backbone of the polymer.

In some forms, Z can be a carboxylate, phosphate, phosphonic, phosphanate, sulfate, sulfinic, or sulfonate. The zwitterionic monomers can be provided in their zwitterionic states, as precursor monomers containing a protecting group, or combinations thereof. After the polymerization reaction, the precursor monomers can be deprotected to produce the zwitterionic monomer. For example, the precursor to a carboxybetaine monomer can be a cationic carboxybetaine ester. After polymerization the cationic carboxybetaine ester is hydrolyzed thereby converting it to the carboxybetaine, i.e., zwitterion.

In some forms, R₆-R₁₈ are independently unsubstituted alkyl, substituted alkyl, unsubstituted alkenyl, substituted alkylene, unsubstituted alkylene, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group.

In some forms, R₆-R₁₈ are independently unsubstituted C₁-C₁₀ alkyl, substituted C₁C₁₀ alkyl, unsubstituted C₁C₁₀ alkenyl, substituted C₁C₁₀ alkylene, unsubstituted C₁-C₁₀ alkylene, substituted C₁-C₁₀ alkenyl, unsubstituted C₁-C₁₀ alkynyl, substituted C₁-C₁₀ alkynyl, unsubstituted aryl, substituted aryl, unsubstituted heteroaryl, substituted heteroaryl, unsubstituted C₁-C₁₀ alkoxy, substituted C₁-C₁₀ alkoxy, unsubstituted aroxy, substituted aroxy, unsubstituted C₁-C₁₀ alkylthio, substituted C₁-C₁₀ alkylthio, unsubstituted arylthio, substituted arylthio, unsubstituted C₁-C₁₀ carbonyl, substituted C₁-C₁₀ carbonyl, unsubstituted C₁-C₁₀ carboxyl, substituted C₁-C₁₀ carboxyl, unsubstituted C₁-C₁₀ amino, substituted C₁-C₁₀ amino, unsubstituted C₁-C₁₀ amido, substituted C₁-C₁₀ amido, unsubstituted C₁-C₁₀ sulfonyl, substituted C₁-C₁₀ sulfonyl, unsubstituted C₁C₁₀ sulfamoyl, substituted C₁C₁₀ sulfamoyl, unsubstituted C₁C₁₀ phosphonyl, substituted C₁-C₁₀ phosphonyl, unsubstituted polyaryl, substituted polyaryl, unsubstituted C₃-C₂₀ cyclic, substituted C₃-C₂₀ cyclic, unsubstituted C₃-C₂₀ heterocyclic, or substituted C₃-C₂₀ heterocyclic.

In some forms, R₆-R₁₈are unsubstituted C₁-C₅ alkyl, substituted C₁-C₅ alkyl, unsubstituted C₁-C₅ alkenyl, substituted C₁-C₅ alkylene, unsubstituted C₁-C₅ alkylene, substituted C₁-C₅ alkenyl, unsubstituted C₁-C₅ alkynyl, substituted C₁-C₅ alkynyl, unsubstituted aryl, substituted aryl, unsubstituted heteroaryl, substituted heteroaryl, unsubstituted C₁-C₅ alkoxy, substituted C₁-C₅ alkoxy, unsubstituted aroxy, substituted aroxy, unsubstituted C₁-C₅ alkylthio, substituted C₁-C₅ alkylthio, unsubstituted arylthio, substituted arylthio, unsubstituted C₁-C₅ carbonyl, substituted C₁-C₅ carbonyl, unsubstituted C₁-C₅ carboxyl, substituted C₁-C₅ carboxyl, unsubstituted C₁-C₅ amino, substituted C₁-C₅ amino, unsubstituted C₁-C₅ amido, substituted C₁-C₅ amido, unsubstituted C₁-C₅ sulfonyl, substituted C₁-C₅ sulfonyl, unsubstituted C₁-C₅ sulfamoyl, substituted C₁-C₅ sulfamoyl, unsubstituted C₁-C₅ phosphonyl, substituted C₁-C₅ phosphonyl, unsubstituted polyaryl, substituted polyaryl, unsubstituted C₃-C₁₀ cyclic, substituted C₃-C₁₀ cyclic, unsubstituted C₃-C₁₀ heterocyclic, or substituted C₃-C₂₀ heterocyclic.

In some forms, R₆, R₉, R_{10,} R₁₁ and R_{15,} are independently unsubstituted C₁-C₅ alkyl, substituted C₁-C₅ alkyl, substituted C₁-C₅ alkylene, or unsubstituted C₁-C₅ alkylene, C₁-C₅ alkoxy, substituted C₁-C₅ alkoxy, unsubstituted aroxy, substituted aroxy, unsubstituted C₁-C₅ alkylthio, substituted C₁-C₅ alkylthio, unsubstituted arylthio, substituted arylthio, unsubstituted C₁-C₅ carbonyl, substituted C₁-C₅ carbonyl, unsubstituted C₁-C₅ carboxyl, substituted C₁-C₅ carboxyl, unsubstituted C₁-C₅ amino, substituted C₁-C₅ amino, unsubstituted C₁-C₅ amido, substituted C₁-C₅ amido, unsubstituted C₁-C₅ sulfonyl, substituted C₁-C₅ sulfonyl, unsubstituted C₁-C₅ sulfamoyl, substituted C₁-C₅ sulfamoyl, unsubstituted C₁-C₅ phosphonyl, or substituted C₁-C₅ phosphonyl.

In some forms, R₇, R₈, R₁₂, R₁₃, R₁₄, R₁₆, R₁₇, and R₁₈, are independently hydrogen, unsubstituted C₁-C₅ alkyl, or substituted C₁-C₅ alkyl.

In some forms, the zwitterionic moieties can be:

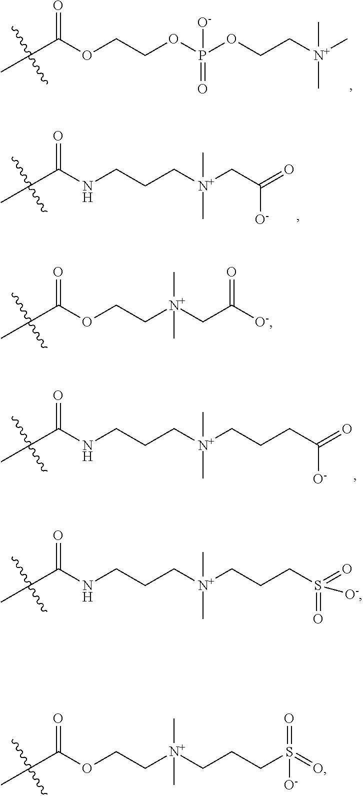

or combinations thereof.

ii. Monomers with a Reactive Side Chain

The zwitterionic polymers optionally contain a monomer, B, with a reactive site chain. The reactive side chain can be represented by the formula:

d-R₁—Y, Formula IV

d is the point of covalent attachment of the reactive side chain to the backbone of the polymer.

In some forms, R₁ is alkyl, substituted alkyl, alkenyl, substituted alkenyl, alkynyl, substituted alkynyl, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), poly(lactic-co-glycolic acid), peptide, or polypeptide group.

In some forms, R₁ is -Aq-unsubstituted C₁-C₁₀ alkylene-Bq-unsubstituted C₁-C₁₀ alkylene-, Aq-unsubstituted C₁-C₁₀ alkylene-Bq-substituted C₁-C₁₀ alkylene-, -Aq-substituted C₁-C₁₀ alkylene-Bq-unsubstituted C₁-C₁₀ alkylene-, or Aq-substituted C₁-C₁₀ alkylene-Bq-substituted C₁-C₁₀ alkylene-, wherein Aq and Bq are independently —C(O)O—, —C(O)NH—, —OC(O)—, —NHC(O)—, —O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

In some forms, R₁ is -Aq-unsubstituted C₁-C₅ alkylene-Bq-unsubstituted C₁-C₅ alkylene-, Aq-unsubstituted C₁-C₅ alkylene-Bq-substituted C₁-C₅ alkylene-, -Aq-substituted C₁-C₅ alkylene-Bq-unsubstituted C₁-C₅ alkylene-, or Aq-substituted C₁-C₅ alkylene-Bq-substituted C₁-C₅ alkylene-, wherein Aq and Bq are independently —C(O)O—, —C(O)NH—, —OC(O)—, —NHC(O)——O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

In some forms, R₁ is —C(O)O-unsubstituted C₂ alkylene—NHC(O)-unsubstituted C₄ alkylene-, —C(O)O-unsubstituted C₂ alkylene-NHC(O)-substituted C₄ alkylene-, —C(O)O-substituted C₂ alkylene—NHC(O)-unsubstituted

C4 alkylene-, or —C(O)O-substituted C2 alkylene—NHC(O)-substituted C₄ alkylene-.

In some forms, Y is propane-1,3-dithiol, 1,2-dithiolan-3-yl, 1,2-dithiol-3-ylidene, amine, hydrogen, —SH, maleimide, aziridine, —N_{3,} —CN, acryloyl, acrylamide, —C(O)OR_{2,} —C(O)R_{3,} vinyl sulfone, —OH, cyanate, thiocyanate, isocyanate, isothiocyanate, alkoxysilane, vinyl silane, silicon hydride, —NR₄R_{5,} acetohydrazide, acyl azide, acyl halides, N-hydroxysuccinimide ester, sulfonyl chloride, glyoxal, epoxide, carbodiimides, aryl halides, imido ester.

In some forms, R₁ is alkyl, substituted alkyl, alkenyl, substituted alkenyl, alkynyl, substituted alkynyl, aryl, substituted aryl, heteroaryl, substituted heteroaryl, alkoxy, aroxy, substituted aroxy, alkylthio, substituted alkylthio, arylthio, substituted arylthio, carbonyl, carboxyl, amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, or polypeptide group; and Y is propane-1,3-dithiol, 1,2-dithiolan-3-yl, 1,2-dithiol-3-ylidene, hydrogen, —SH, maleimide, aziridine, —N_{3,} —CN, acryloyl, acrylamide, —C(O)OR_{2,} —C(O)R_{3,} vinyl sulfone, —OH, cyanate, thiocyanate, isocyanate, isothiocyanate, alkoxysilane, vinyl silane, silicon hydride, —NR₄R_{5,} acetohydrazide, acyl azide, acyl halides, N-hydroxysuccinimide ester, sulfonyl chloride, glyoxal, epoxide, carbodiimides, aryl halides, imido ester, or

R₁ is alkyl, substituted alkyl, alkenyl, substituted alkenyl, alkynyl, substituted alkynyl, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), poly(lactic-co-glycolic acid), peptide, or polypeptide group; and Y is propane-1,3-dithiol, 1,2-dithiolan-3-yl, 1,2-dithiol-3-ylidene, —SH, maleimide, aziridine, —N_{3,} —CN, acrylamide, —C(O)OR_{2,} —C(O)R_{3,} vinyl sulfone, cyanate, thiocyanate, isocyanate, isothiocyanate, vinyl silane, silicon hydride, acetohydrazide, acyl azide, acyl halides, N-hydroxysuccinimide ester, sulfonyl chloride, glyoxal, carbodiimides, aryl halides, imido ester.

In some forms, R_{2,} R_{4,} and R_{5,} are, independently, hydrogen, amino, hydroxyl, thiol, oxo, phosphate, or substituted or unsubstituted C₁-C₁₀ alkyl, C₁-C₁₀ alkylene, C₂-C₁₀ alkenyl, C₂-C₁₀ alkynyl, C₁-C₁₀ alkoxy, C₁-C₁₀ alkylamino, C₁-C₁₀ alkylthio, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, or substituted heterocyclic; and

wherein R₃ is hydrogen, amino, hydroxyl, thiol, oxo, phosphate, or substituted or unsubstituted C₁-C₁₀ alkyl, C₁-C₁₀ alkylene, C₂-C₁₀ alkenyl, C₂-C₁₀ alkynyl, C₁-C₁₀ alkoxy, C₁-C₁₀ alkylamino, C₁-C₁₀ alkylthio, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, or substituted heterocyclic.

iii. Hydrophobic Monomer

The polymers optionally contain a hydrophobic monomer with a hydrophobic side chain, represented by:

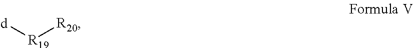

d is the point of covalent attachment of the hydrophobic side chain to the backbone of the polymer.

In some forms of Formula V, R₁₉ and R₂₀ are independently unsubstituted alkyl, substituted alkyl, unsubstituted alkenyl, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, —O—, —S—, —NH—NHC(O)—, —N═N—, —N═CH—, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group.

In some forms of Formula V, R₁₉ is —C(O)NH—, —C(O)O—, —NHC(O)—, —OC(O)—,

—O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

In some forms of Formula V, R₂₀ has the structure:

-Az-Bz-(—C_{z})δ, Formula VII

wherein δ is an integer between 0 and 10, inclusive, preferably δ is 1.

In some forms of Formula VII, Az can be:

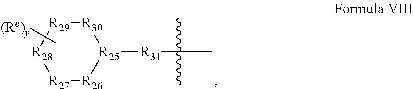

wherein R₃₁ in Az is —(CR₃₂R₃₂)ₚ—; p is an integer from 0 to 5; each R₃₂ is hydrogen, unsubstituted alkyl, or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R_{25,} R_{26,} R_{27,} R_{28,} R_{29,} and R₃₀ are independently C or N, wherein the bonds between adjacent R₂₅ to R₃₀ are double or single according to valency, and wherein R₂₅ to R₃₀ are bound to none, one, or two hydrogens according to valency.

In some forms of Formula VIII, each R₃₂ is hydrogen, and p is 1.

In some forms of Formula VIII, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds.

In some forms of Formula VIII, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, and y is 1.

In some forms of Formula VIII, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, y is 1, and R^{e} is Bz.

In some forms of Formula VIII, each R₃₂ is hydrogen, p is 1, R₂₅ is C, and R₂₆-R₃₀ are CH, and the bonds between R₂₅ and R_{26,} between R₂₇ and R_{28,} and between R₂₉ and R₃₀ are double bonds, y is 1, and R^{e} contains a substituted heteroaryl group.

In some forms of Formula VIII, each R₃₂ is hydrogen, p is 1, R₂₅ is C, and R₂₆-R₃₀ are CH, and the bonds between R₂₅ and R₂₆, between R₂₇ and R₂₈, and between R₂₉ and R₃₀ are double bonds, y is 1, R^{e} contains a substituted heteroaryl group, wherein the substituted heteroaryl group is a substituted triazole.

In some forms of Formula VII, Az can be:

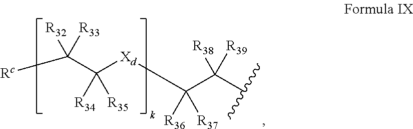

wherein R₃₂, R₃₃, R₃₄, R₃₅, R₃₆, R₃₇, R₃₈, and R₃₉ in Az are independently hydrogen, unsubstituted alkyl, substituted alkyl, unsubstituted alkenyl, substituted alkenyl, unsubstituted alkynyl, substituted alkynyl, unsubstituted phenyl, substituted phenyl, unsubstituted aryl, substituted aryl, unsubstituted heteroaryl, substituted heteroaryl, unsubstituted arylalkyl, substituted arylalkyl, unsubstituted alkoxy, substituted alkoxy, unsubstituted aroxy, substituted aroxy, unsubstituted carbonyl, substituted carbonyl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, substituted C₃-C₂₀ heterocyclic, poly(ethylene glycol), or poly(lactic-co-glycolic acid); k is an integer from 0 to 20; each X_{d} is independently absent, O, or S; and R^{c} can be Bz.

In some forms of Formula IX, Xd is O. In some forms of Formula IX, Xd is O, and R₃₂-R₃₉ are hydrogen.

In some forms of Formula IX, Xd is O, R₃₂-R₃₉ are hydrogen, and k is an integer between 1 and 5, inclusive, preferably 3.

In some forms of Formula VII or IX, Bz can be:

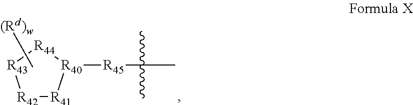

wherein R₄₅ in Bz is —(CR₄₆R₄₆)ₚ—; p is an integer from 0 to 5; each R₄₆ is hydrogen, unsubstituted alkyl, or substituted alkyl; each R^{d} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; w is an integer between 0 and 4, inclusive; each R_{40,} R_{41,} R_{42,} R_{43,} and R_{44,} are independently C or N, wherein the bonds between adjacent R₄₀ to R₄₄ are double or single according to valency, and wherein R₄₀ to R₄₄ are bound to none, one, or two hydrogens according to valency.

In some forms of Formula X, p is 0.

In some forms of Formula X, p is 0, and R₄₀-R₄₂ are N.

In some forms of Formula X, p is 0, R₄₀-R₄₂ are N, and R₄₃ and R₄₄ are C.

In some forms, Formula X is:

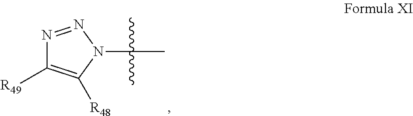

wherein R₄₈ and R₄₉ are independently hydrogen,

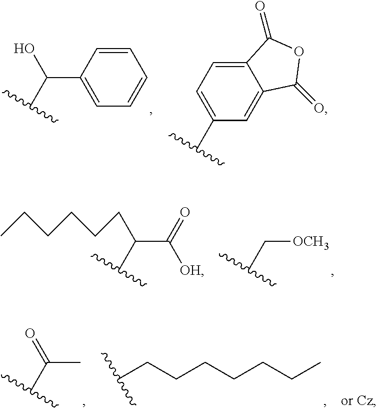

with the proviso that at least one of R₄₈ and R₄₉ is not hydrogen.

In some forms of Formula VII or Formula XI, Cz can be:

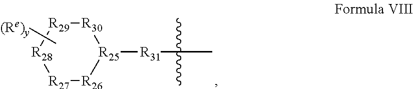

wherein R₃₁ in Cz is —(CR₃₂R₃₂)ₚ— or —(CR₃₂R₃₂)ₚ—X_{b}—(CR₃₂R₃₂)_{q}—; p and q are independently integers between 0 to 5, inclusive; each R₃₂ is hydrogen, unsubstituted alkyl, or substituted alkyl; X_{b} is absent, O, —S—, —S(O)—, —S(O)₂—, or NR_{47;} R₄₇ is unsubstituted alkyl or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R_{25,} R₂₆, R_{27,} R₂₈, R_{29,} and R₃₀ are independently C or N, wherein the bonds between adjacent R₂₅ to R₃₀ are double or single according to valency, and wherein R₂₅ to R₃₀ are bound to none, one, or two hydrogens according to valency.

In some forms of Formula VIII, R₃₁ is —(CR₃₂R₃₂)ₚ—, each R₃₂ is hydrogen, and p is 1.

In some forms of Formula VIII, R₃₁ is —(CR₃₂R₃₂)ₚ—, each R₃₂ is hydrogen, p is 1, and R₂₅ is N.

In some forms of Formula VIII, R₃₁ is —(CR₃₂R₃₂)ₚ—, each R₃₂ is hydrogen, p is 1, R₂₅ is N, and R₂₈ is S(O)₂.

In some forms of Formula VIII, R₃₁ is —(CR₃₂R₃₂)ₚ—, each R₃₂ is hydrogen, p is 1, R₂₅ is N, R₂₈ is S(O)_{2,} and R₂₆, R_{27,} R_{29,} and R₃₀ are CH₂.

In some forms of Formula VIII, R₃₁ is —(CR₃₂R₃₂)ₚ—, each R₃₂ is hydrogen, p is 1, R₂₅ is N, R₂₈ is S(O)_{2,} R₂₆, R_{27,} R_{29,} and R₃₀ are CH_{2,} and y is 0, i.e.,

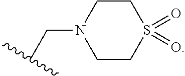 [SMILES: *CN1CCS(=O)(=O)CC1]

In some forms of Formula VIII, R₃₁ is —(CR₃₂R₃₂)ₚ—X_{b}—(CR₃₂R₃₂)_{q}—, each R₃₂ is hydrogen, and p is 0.

In some forms of Formula VIII, R₃₁ is —(CR₃₂R₃₂)ₚ—X_{b}—(CR₃₂R₃₂)_{q}—, each R₃₂ is hydrogen, p is 0, and q is 1.

In some forms of Formula VIII, R₃₁ is —(CR₃₂R₃₂)ₚ—X_{b}—(CR₃₂R₃₂)_{q}—, each R₃₂ is hydrogen, p is 0, q is 1, and X_{b} is O or —S(O)₂—.

In some forms of Formula VIII, R₃₁ is —(CR₃₂R₃₂)ₚ—X_{b}—(CR₃₂R₃₂)_{q}—, each R₃₂ is hydrogen, p is 0, q is 1, X_{b} is O, and R₂₆ is O.

In some forms of Formula VIII, R₃₁ is —(CR₃₂R₃₂)ₚ—X_{b}—(CR₃₂R₃₂)_{q}—, each R₃₂ is hydrogen, p is 0, q is 1, X_{b} is O, R₂₆ is O, and R₂₅ is CH.

In some forms of Formula VIII, R₃₁ is —(CR₃₂R₃₂)ₚ-X_{b} —(CR₃₂R₃₂)_{q}-, each R₃₂ is hydrogen, p is 0, q is 1, X_{b} is O, R₂₆ is O, R₂₅ is CH, R₂₇-R₃₀ are CH_{2,} and y is 0, i.e.,

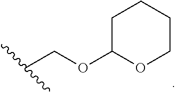 [SMILES: *COC1CCCCO1]

In some forms of Formula VIII, R₃₁ is —(CR₃₂R₃₂)ₚ—X_{b}—(CR₃₂R₃₂)_{q}—, each R₃₂ is hydrogen, p is 0, q is 1, X_{b} is —S(O)₂—, R₂₅ is C, R₂₆-R₃₀ are CH, and the bonds between R₂₅ and R₂₆, between R₂₇ and R₂₈, and between R₂₉ and R₃₀ are double bonds, i.e.,

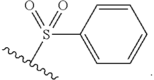 [SMILES: *S(=O)(=O)c1ccccc1]

In some forms of Formula VIII, R₃₁ is —(CR₃₂R₃₂)ₚ—, and p is 0.

In some forms of Formula VIII, R₃₁ is —(CR₃₂R₃₂)ₚ—, p is 0, R₂₅ is C, and R₂₆-R₃₀ are CH, and the bonds between R₂₅ and R₂₆, between R₂₇ and R₂₈, and between R₂₉ and R₃₀ are double bonds.

In some forms of Formula VIII, R₃₁ is —(CR₃₂R₃₂)ₚ—, p is 0, R₂₅ is C, and R₂₆-R₃₀ are CH, the bonds between R₂₅ and R₂₆, between R₂₇ and R₂₈, and between R₂₉ and R₃₀ are double bonds, and y is 0 or 1.

In some forms of Formula VIII, R₃₁ is —(CR₃₂R₃₂)ₚ—, p is 0, R₂₅ is C, and R₂₆-R₃₀ are CH, the bonds between R₂₅ and R₂₆, between R₂₇ and R₂₈, and between R₂₉ and R₃₀ are double bonds, y is 1, and R^{e} is —NH₂, —OCH₃, or —CH₂OH, i.e.,

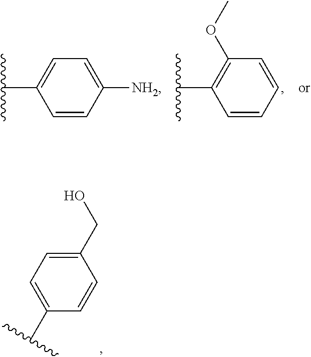

respectively.

In some forms of Formula VIII, R₃₁ is —(CR₃₂R₃₂)ₚ—, p is 0, R₂₅ is C, R₂₇ is N, R₂₆, R₂₈-R₃₀ are CH, the bonds between R₂₅ and R₂₆, between R₂₇ and R_{28,} and between R₂₉ and R₃₀ are double bonds, and y is 0, i.e.,

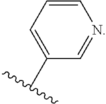 [SMILES: *c1cccnc1]

In some forms of Formula VIII, R₃₁ is —(CR₃₂R₃₂)_{p—, p is} 0, R₂₅ is C(OH), and R₂₆-R₃₀ are CH_{2,} and y is 0, i.e.,

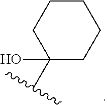 [SMILES: *C1(O)CCCCC1]

In some forms, the hydrophobic monomeric unit contains the moiety:

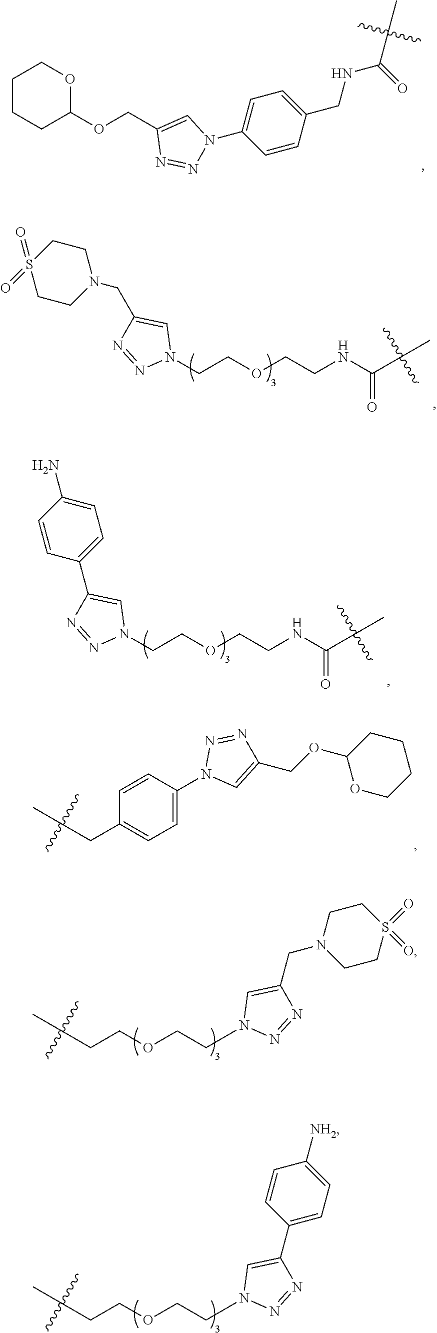

or combinations thereof.

iv. Neutral Hydrophilic Monomer

The polymers optionally contain a neutral hydrophilic monomer with a hydrophilic side chain represented by:

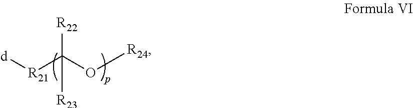

d is the point of covalent attachment of the neutral hydrophilic side chain to the backbone of the polymer.

p is an integer between 1 and 10,000, inclusive, preferably between 1 and 30, inclusive.

In some forms, R₂₁ is unsubstituted alkyl, substituted alkyl, unsubstituted alkenyl, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group.

In some forms, R_{22,} R_{23,} and R₂₄ are independently hydrogen, alkyl, substituted alkyl, alkenyl, substituted alkenyl, alkynyl, substituted alkynyl, aryl, substituted aryl, heteroaryl, substituted heteroaryl, alkoxy, aroxy, substituted aroxy, alkylthio, substituted alkylthio, arylthio, substituted arylthio, carbonyl, carboxyl, amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, or polypeptide group.

In some forms, R₂₁ is a substituted carbonyl, R_{22,} R_{23,} and R₂₄ are hydrogen, and p is an integer between 1 and 20, inclusive.

In some forms, R₂₁ is a substituted carbonyl, R₂₂ and R₂₃ are hydrogen, R₂₄ is methyl, and p is an integer between 1 and 1000, inclusive.

In some forms, the macrodevice contains a polymer that contains a structure selected from:

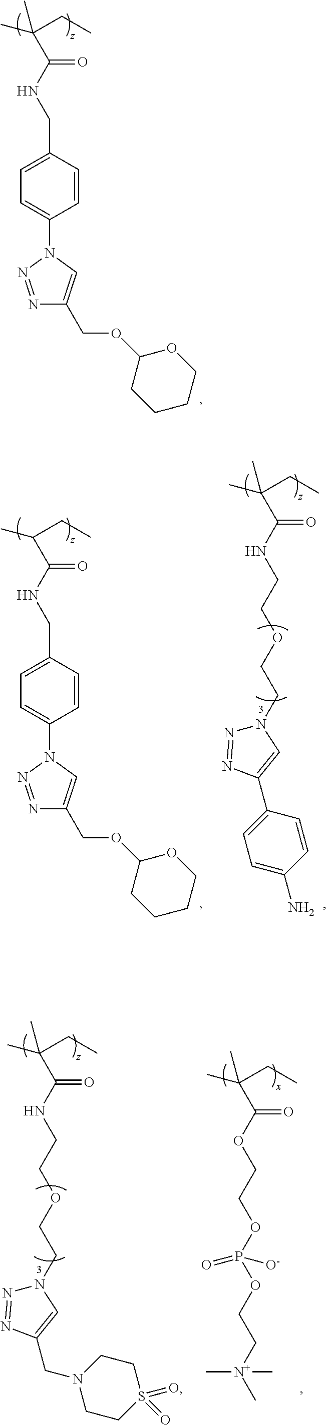

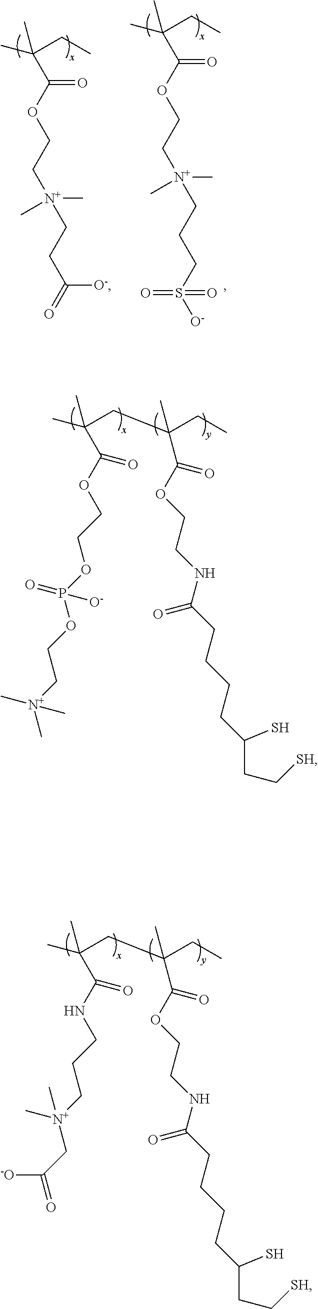

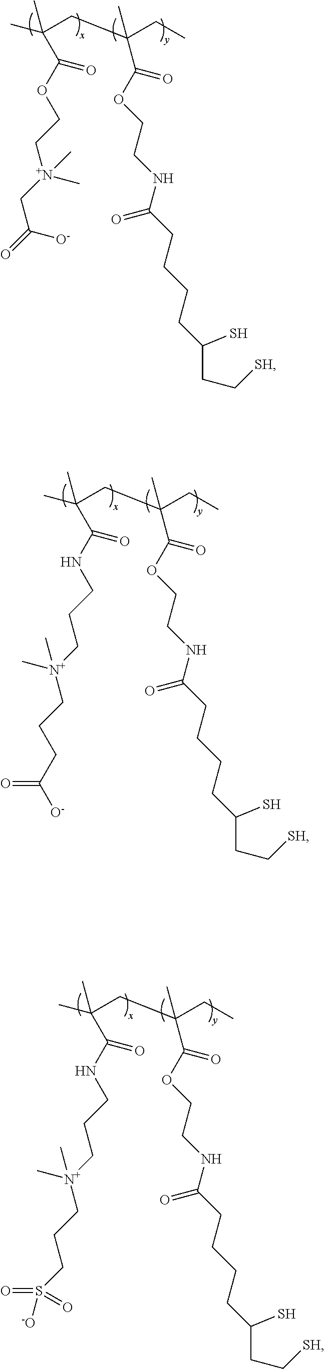

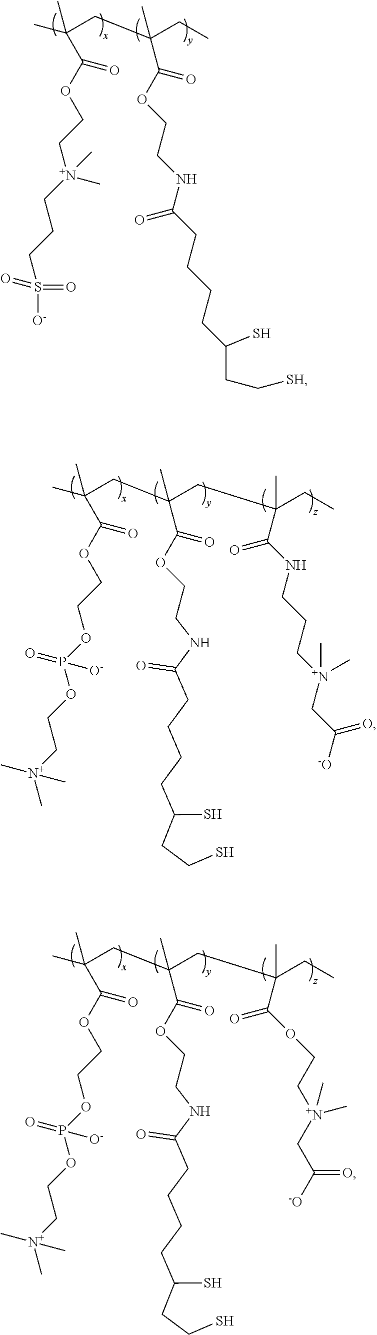

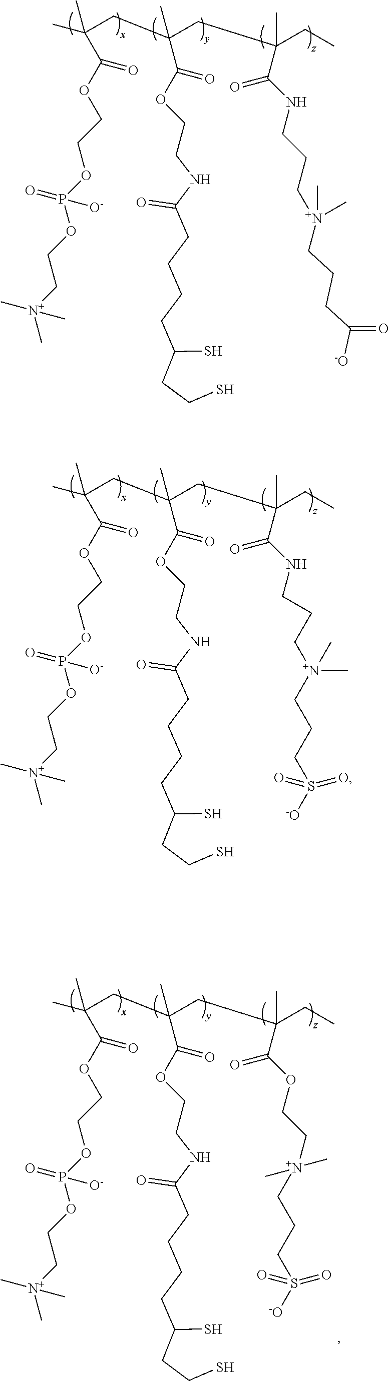

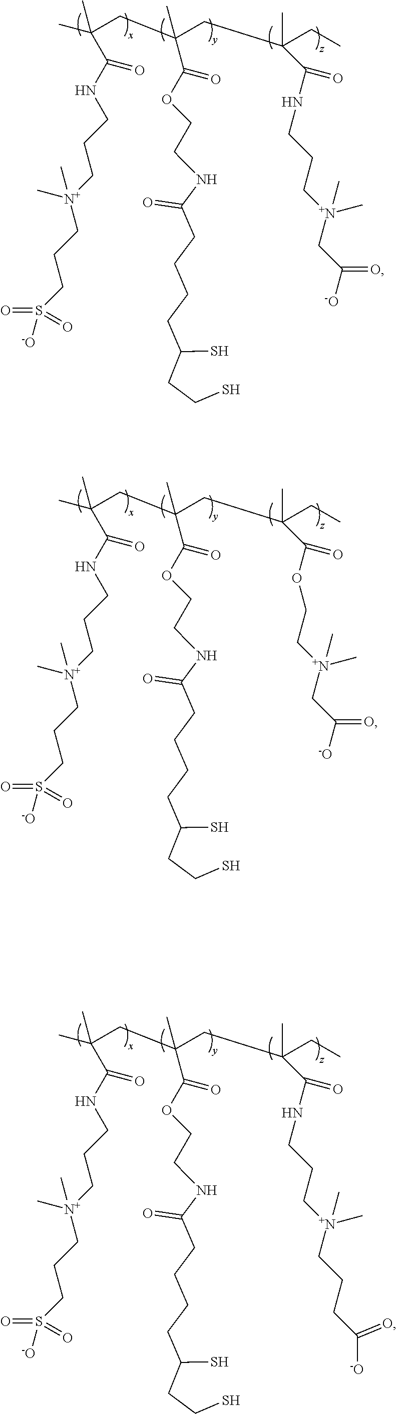

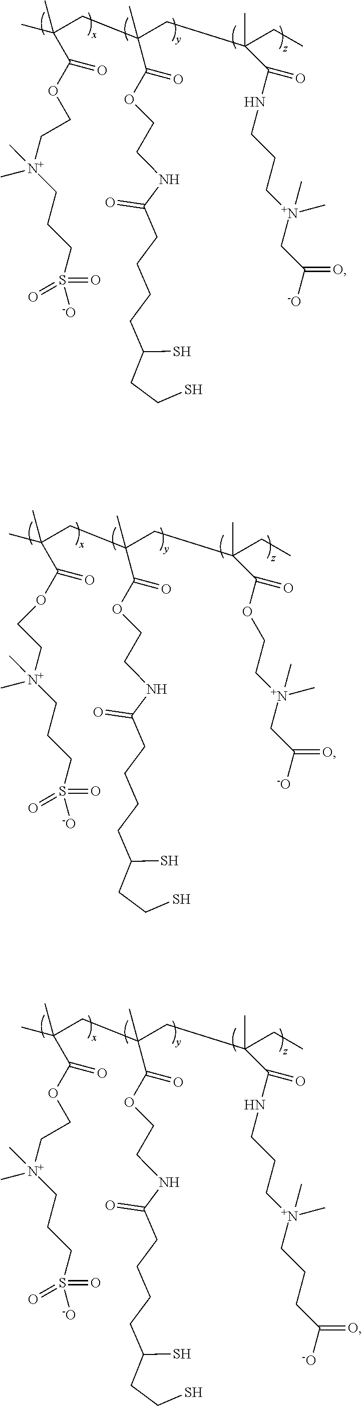

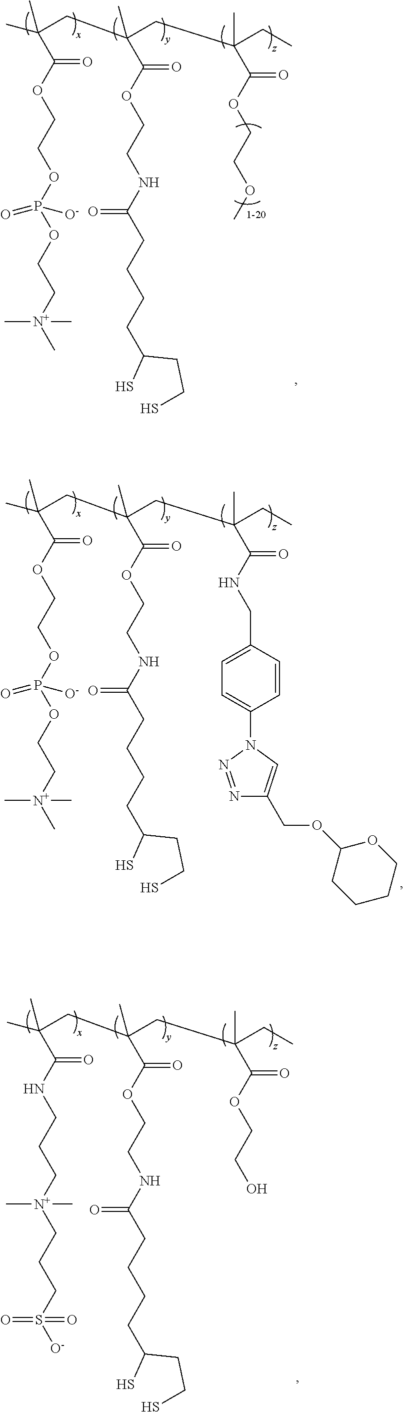

or a combination thereof,

wherein, x and y are independently integers between 1 and 1000, inclusive, preferably x is between 10 and 200, inclusive, preferably y is between 2 and 20, inclusive; and

z is between 0 and 1000, inclusive, preferably z is between 10 and 200, inclusive.

Other polymers can used to modify the surface of the macrodevices described herein. These include chemically modified alginates such as those described in WO 2016/019391, WO 2017/075631, US 2016/0324793, and WO 2012/167223, the contents of which are incorporated herein by reference.

4. Weight Average Molecular Weight

The weight average molecular weight of the polymers can vary. In some forms, the weight average molecular weight of the polymer, as determined by size exclusion chromatography (SEC), can be between about 500 Daltons and about 50,000 Daltons, preferably between about 2,000 Daltons and about 30,000 Daltons, most preferably between about 5,000 Daltons and about 20,000 Daltons. The weight average molecular weights of the polymers can also depend on their degree of polymerization. In some forms, degree of polymerization is between about 2 and about 10,000, inclusive, between about 2 and about 5,000, inclusive, between about 5 and about 1,000, inclusive, between about 5 and about 500, inclusive, between about 10 and about 200, inclusive, or between about 20 and about 80, inclusive.

In some forms, the small molecules and/or polymers are used to chemically modify the surface of the macrodevices devices described herein. Preferably, small molecules and/or polymers can reduce fibrosis of the macrodevices after implantation into a subject, compared to a corresponding macrodevice whose surface is chemical modified with the small molecules and/or polymers described herein. The polymers can be homopolymers, block copolymers, or random copolymers.

III. Methods of Making

A. Macrodevices

The macrodevices can be fabricated using micro-fabrication techniques known in the art. Exemplary micro-fabrication techniques include, but are not limited to photolithography, soft lithography, microcontact printing, injection molding, embossing etc. The methods have been reviewed in Bettinger, et al., in Nanotechnology and Tissue Engineering CRC Press 2008, pages 87-119, Qin, et al., Topics in Current Chemistry 1998, 194, 1-19, Voldman, et al., Annu. Rev. Biomed. Eng. 1999, 01, 401-425, and Koch, et al., Materials 2016, 9, 646, the contents of which are incorporated herein by reference.

B. Small Molecules

Small molecules that can be used to modify the surfaces of the products include all of the small molecules. Useful small molecules include, but are not limited to, alcohols, thiols, amines, and combinations thereof.

1. Alcohols

Preferred alcohols for use as reagents in esterification include those shown below.

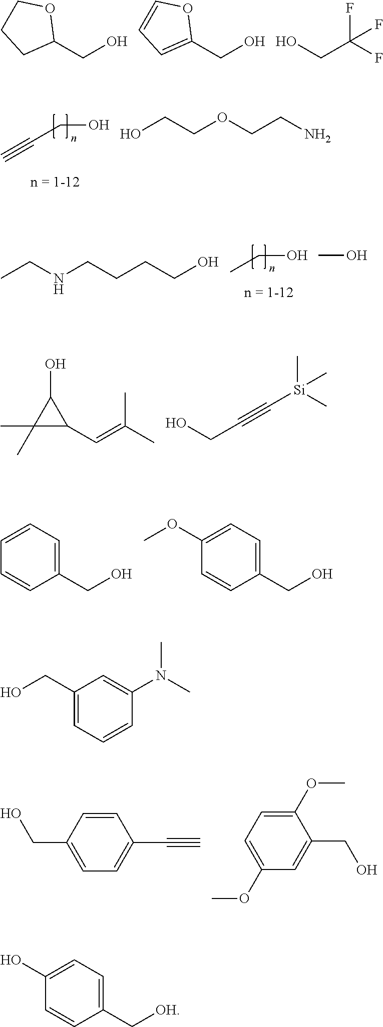

2. Amines

Preferred amines that can be used to modify the surfaces of the products include, but are not limited to,

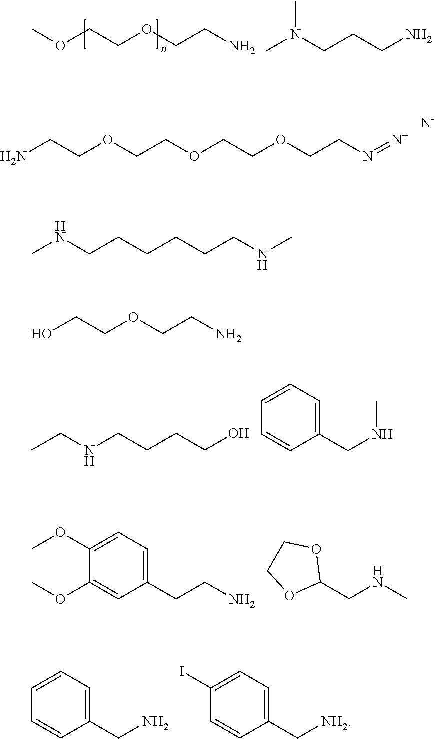

3. Derivatization Via Click Chemistry

In some embodiments, the surfaces of the macrodevices are covalently modified initially to introduce a functional group which can be further reacted via click chemistry.

In preferred embodiments, the alcohols, amines or thiols are used to introduce a functional group which can further reacted using a 1,3-dipolar cycloaddition reaction (i.e., a Huisgen cycloaddition reaction). In a 1,3-dipolar cycloaddition reaction, a first molecule containing an azide moiety is reacted with a second molecule containing a terminal or internal alkyne. As shown below, the azide and the alkyne groups undergo an intramolecular 1,3-dipolar cycloaddition reaction, coupling the two molecules together and forming a 1,2,3-triazole ring.

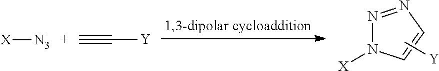

The regiochemistry of 1,3-dipolar cycloadditions reaction can be controlled by addition of a copper(I) catalyst (formed in situ by the reduction of CuSO₄ with sodium ascorbate) or a ruthenium catalyst (such as Cp*RuCl(PPh₃)_{2,} Cp*Ru(COD), or Cp*[RuCl₄]). For example, using a copper catalyst, azides and terminal alkynes can be reacted to exclusively afford the 1,4-regioisomers of 1,2,3-triazoles. Similarly, in the presence of a suitable ruthenium catalyst, azides can be reacted with internal or terminal alkynes to form exclusively the 1,5-regioisomers of 1,2,3-triazoles.

In some forms, the alcohol, amine or thiol containing an alkyne moiety is used to modify the surface initially. In these forms, the alkyne moiety present on the surface can be further reacted with a second molecule containing an azide functional group. Upon reaction, the azide and the alkyne groups undergo an intramolecular 1,3-dipolar cycloaddition reaction forming a 1,2,3-triazole ring, coupling the second molecule to the covalently modified surface.

In some forms, the small molecules are synthesized and these used post-synthesis to modify the surface of the macrodevices. In some forms, the small molecules are synthesized using the alcohols, amines, and alkynes described herein. Examples of small molecules that can be used to modify the surface of the macrodevices include, but are not limited to molecules that contain a substituted heteroaryl group such as,

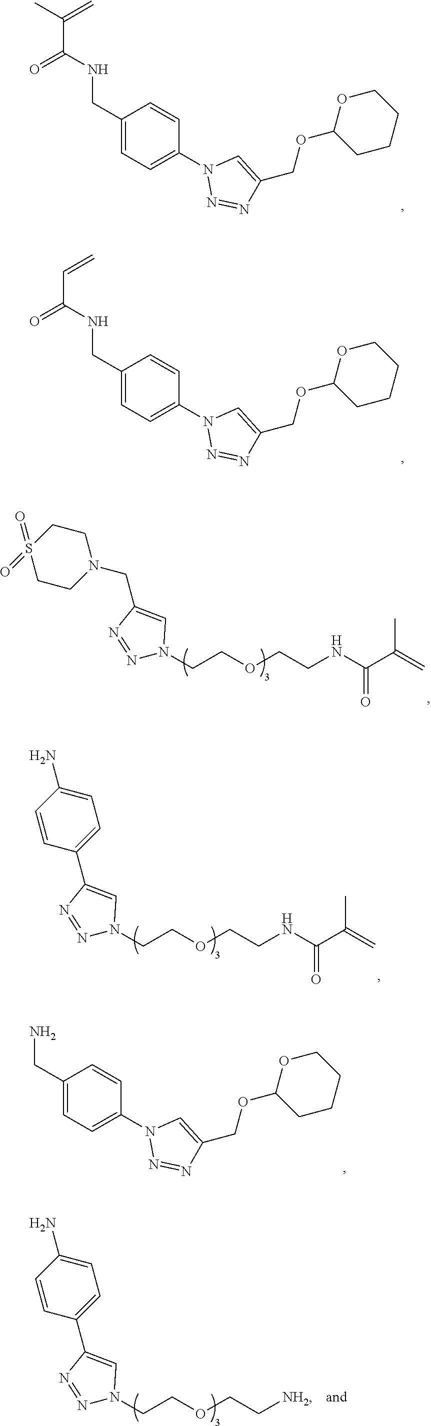

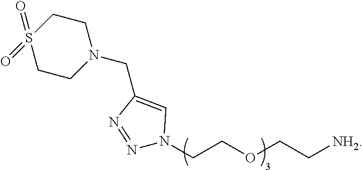

C. Polymers

The polymers can be synthesized using any of the small molecule or chemical moieties containing any of the monomeric units described herein, using polymerization methods known in the art, such as reversible addition-fragmentation chain transfer, and atom transfer radical polymerization. Preferably, the polymers are formed in situ on the surface of the device, using a surface-initiated atom transfer radical polymerization (si-ATRP).

D. Coated Macrodevices

The polymers and/or small molecules described herein for chemically modifying a surface of the macrodevices can be covalently (directly or indirectly) or non-covalently associated with a surface of the macrodevices. Preferably, the polymers and/or small molecules are covalently associated with a surface of the macrodevices.

Covalently association can be performed with the polymers, by grafting molecules on a surface of the macromolecules via a method such as si-ATRP. In some forms, prior to chemically modifying a surface of the macrodevices, the surface can be derivatized by treating the surface with plasma. In some forms, the surfaces of the macrodevices can be treated with a material, such as another polymer, followed by the polymers and/or small molecules onto the treated surface. As a non-limiting example, the surface of the macrodevice can be modified first with mussel-inspired polydopamine (PDA) films by oxidative self-polymerization of dopamine, and followed by conjugation of the polymers and/or small molecules to the PDA film via any reactive group in the reactive side chains of the polymer or small molecule, such as thiol or amine. It has been previously shown that simple immersion of virtually any substrate in an alkaline aqueous solution of dopamine results in spontaneous deposition of a thin PDA film, with subsequent reactivity of this film toward amines and thiols to form ad-layers (Lee, et al., Science 2007, 318, 426; Lee, et al., Adv. Mater. 2009, 21, 431; and Ham, et al., Angew. Chem. Int. Ed. 2011, 50, 732).

The surfaces of macrodevices can be chemically modified as described herein to any desired density of modifications. The density of modifications is the average number of modifications (that is, attached compounds) per a given area of the surface or a surface of the product. Generally, a density at or above a threshold density can provide a beneficial effect, such as lower foreign body response. In some embodiments, a high density is not required. Without being bound to any particular theory of operation, it is believed that the chemical modifications signal to, indicate to, or are identified by, one or more immune system or other body components to result in a beneficial effect, such as a lower foreign body response. In some embodiments, a lower density of modifications can be effective for this purpose.

Useful densities include densities of at least, of less than, of about, or of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 320, 340, 360, 380, 400, 420, 440, 460, 480, 500, 550, 600, 650, 700, 750, 800, 850, 900, and 1000 modifications per square μm. All ranges defined by any pair of these densities are also specifically contemplated and disclosed.

In some embodiments, the density of the modifications on a surface, surfaces, or portions of a surface(s) of a product that, when the product is administered to (e.g., implanted in the body of) a subject, would be in contact with fluid(s), cell(s), tissue(s), other component(s), or a combination thereof of the subject's body is greater than the density of the modifications on other surfaces of the product.

Density can also be expressed in terms of the concentration of the surface modifications as measured by X-ray photoelectron spectroscopy (XPS). XPS is a surface-sensitive quantitative spectroscopic technique that measures the elemental composition at the parts per thousand range of the elements that exist within a material. XPS spectra are obtained by irradiating a material with a beam of X-rays while simultaneously measuring the kinetic energy and number of electrons that escape from the top 0 to 10 nm of the material being analyzed. By measuring all elements present on the surface, the percentage of the elements that come from the surface modifications can be calculated. This can be accomplished by, for example, taking the percentage of nitrogen (and/or other elements in the surface modifications) in the total elemental signal measured. Nitrogen is a useful indicator for the surface modification because many substrated and materials forming the product contain little nitrogen. For convenience, the percent of the element(s) used to indicate the surface modifications can be stated as the percent surface modifications. Also for convenience, the percent surface modifications can be referred to as the concentration of surface modifications. Examples of XPS analysis and concentrations of surface modifications are shown in Tables 4-7.

Useful percent surface modifications include concentrations of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 percent surface modifications. All ranges defined by any pair of these concentrations are also specifically contemplated and disclosed.

IV. Methods of Using

The macrodevices described herein can be used in applications where improved performance, such as reduced fibrosis and/or long-term delivery of a therapeutic agent to a subject in need thereof. These include, but are not limited, implanting encapsulated cells, such as engineered cells, stem cell-derived cells to secrete therapeutic agents in cell-based therapies. Therapeutic agents can be hormones (e.g. insulin, erythropoietin, etc.), growth factors, cytokines, co-factors, secretory proteins, structural proteins, etc.

The methods, compounds, and compositions herein described are further illustrated in the following examples, which are provided by way of illustration and are not intended to be limiting. It will be appreciated that variations in proportions and alternatives in elements of the components shown will be apparent to those skilled in the art and are within the scope of disclosed forms. All parts or amounts, unless otherwise specified, are by weight.

### EXAMPLES

### Example 1

### Biocompatible Macrodevices for Transplanting Cells

### Materials and Methods

The body of a macrodevice was micro-fabricated using poly(dimethylsiloxane) (PDMS). A polycarbonate track-etched membrane was attached to one side, the top side, of a PDMS micro-fabricated body using aminosilane chemistry. Different macrodevices were generated by attaching a porous membrane with pore size of 0.4 μm, 0.6 μm, 0.8 μm, 1.0 μm, or 3.0 μm to the micro-fabricated body. A human cell line (HEK 293) was engineered to secrete a cytokine mouse-erythropoietin (EPO), and cells from this cell line were encapsulated in one or more compartments in the micro-fabricated body of the macrodevices with these different porous membranes and the macrodevices were then transplanted into the IP space of Balb/c mice. Serum EPO levels in the mice were monitored.

After five weeks, implanted macrodevices containing porous membranes of various pore sizes were retrieved from IP space of blab/c mice, and stained for actin, macrophages, T-cells (CD8 cells), and HEK cell. The stained macrodevices were analyzed via confocal imaging.

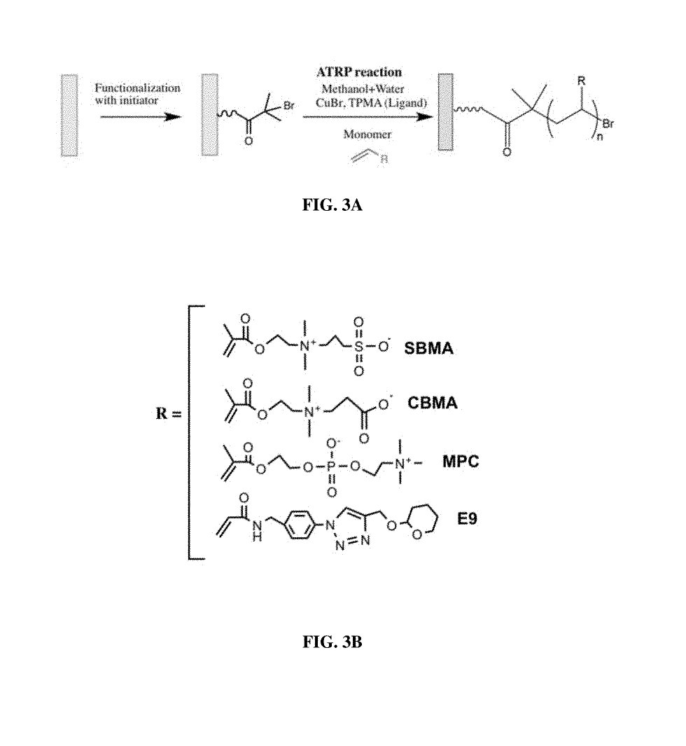

Surface-initiated atom transfer radical polymerization (si-ATRP) was used to grow four polymer grafts on the surface of the macrodevice, FIGS. 3A and 3B. The polymers were synthesized using one of three zwitterionic monomers belonging to the major classes of zwitterionics or a monomer with a hydrophobic side chain N-(4-(4-(((tetrahydro-2H-pyran-2-yl)oxy)methyl)-1H-1,2,3-triazol-1-yl)benzyl)acrylamide (THPT) (E9). The chemically modified surfaces of the macrodevices were analyzed using X-ray photoelectron spectroscopy (XPS) for the presence of nitrogen. EPO producing HEK cells were encapsulated in these macrodevices with different coatings and implanted IP space of C57BL/6 mice. The macrodevices were retrieved at four weeks post-implantation and analyzed for fibrotic content.

Further, E9-coated macrodevices encapsulating the EPO-secreting cell line described above were implanted in C57BL/6 mice and protein production was monitored during this time period.

### Results

Porous membranes with several pore sizes were tested for their ability to inhibit diffusion of immune cells into the compartments within the micro-fabricated body of the macrodevices. The pore sizes ranged between 0.4 μm and 3.0 μm, inclusive. It was found that the pore size determined the type of immune cells that can infiltrate the device. Implanted devices in the intraperitoneal (IP) space of Balb/c mice with pore size of 3 μm led to infiltration of T-cells and macrophages, which led to destructions of the encapsulated cells. A pore size of 1 micron selectively allowed macrophage infiltration without causing any harm to the encapsulated cells. When implanted in Balb/c mice, a pore size of 0.8 μm, and lower than 0.8 μm, prevented infiltration of immune cells into the compartments within the micro-fabricated body of the macrodevices. This was shown by the level of EPO in the serum of mice monitored for five weeks, FIG.2.

After this five-week period, confocal imaging showed substantial infiltration in macrodevices containing porous membranes with pore sizes of 3 μm and 1 μm, but not in macrodevices having porous membranes with pore sizes of 0.8 μm. Macrodevices with porous membranes having pore sizes of 0.8 μm or less were preferred.

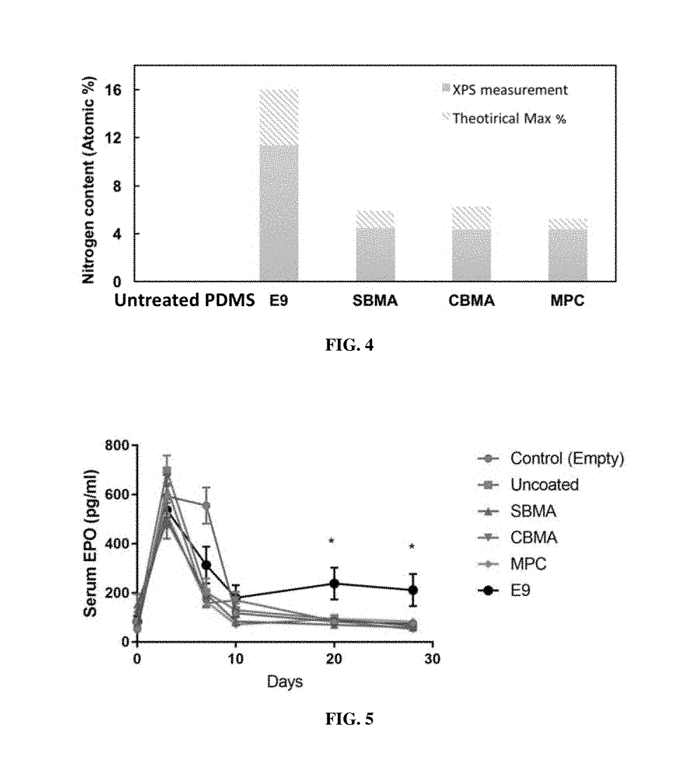

si-ATRP was used to graft polymers on the surface of the macrodevices. The XPS analysis for nitrogen content demonstrated that the polymers were successfully grafted, and also showed a high degree of surface coverage of these polymers, FIG. 4.

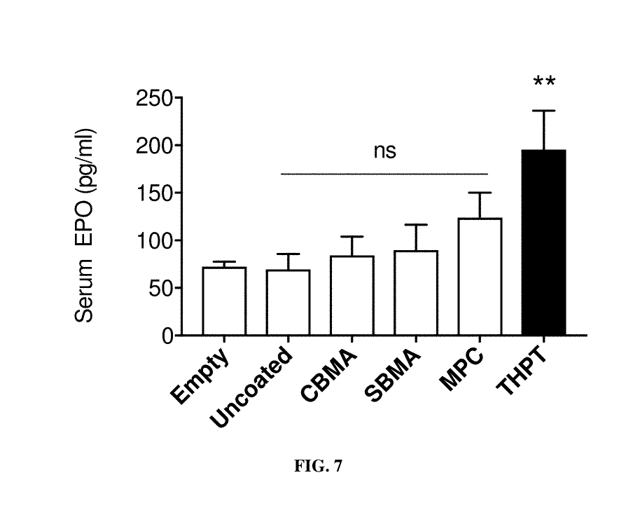

EPO producing HEK cells were encapsulated in the device with different coatings and implanted IP space of C57BL/6 mice. Upon retrieval at 4 weeks, macrodevices with coatings formed from polymers containing only the zwitterionic monomers were heavily fibrosed, but not the macrodevices coated with E9 as shown by macroscopic images of the retrieved devices. In addition, only the mice implanted with E9 coated macrodevices had significantly higher serum EPO levels indicating improved functioning of macrodevices with the E9 coating, FIG. 5 and FIG. 7.

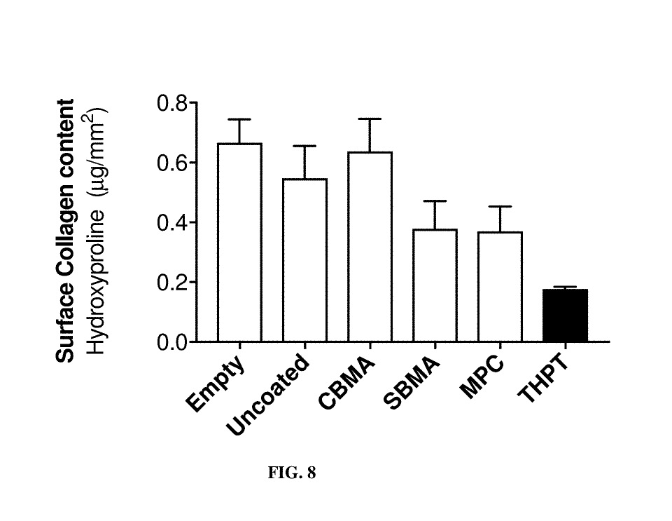

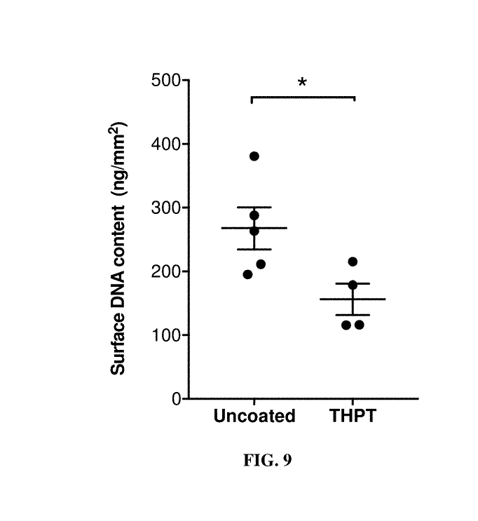

Hydroxyproline assay was used to measure surface concentration of collagen on the devices which shows that the THPT (E9) coated devices had the lowest amount of collagen on their surface, FIG. 8. n=5 per group. One way Anova with Bonferroni correction for multiple comparison was used to compare groups. *p<0.05, **p<0.01.

E9 coating reduces cellular attachment to the surface of the devices. Total DNA from cells attached to devices was extracted from the retrieved device a comparison was made between coated and uncoated devices, FIG. 9. n>=4. Two failed t-test p=0.035 (*)

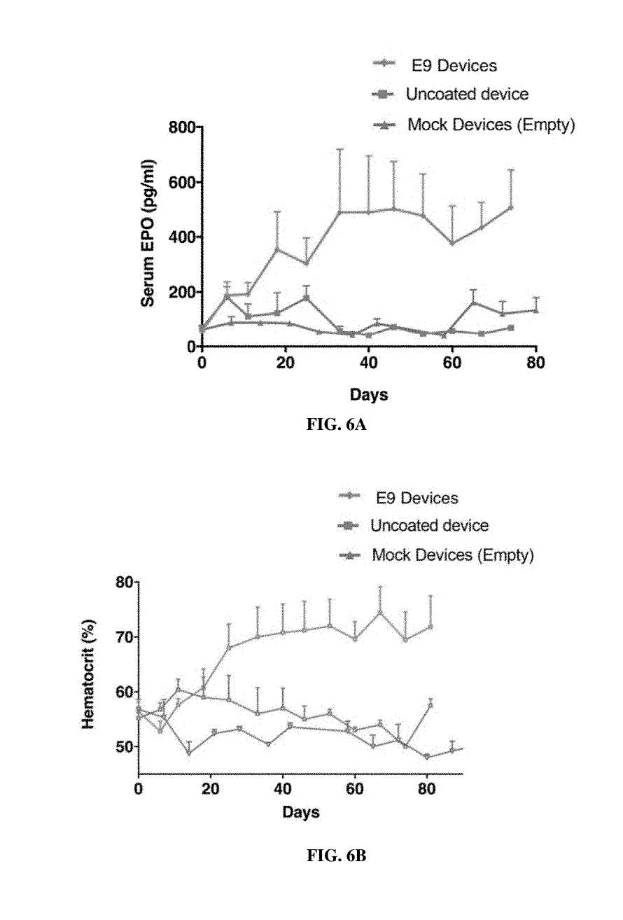

E9 coated macrodevices encapsulating EPO-secreting cells maintained high protein production rates in in C57BL/6 mice, compared to control (uncoated) macrodevices and mock (empty) devices, FIGS. 6A and 6B.

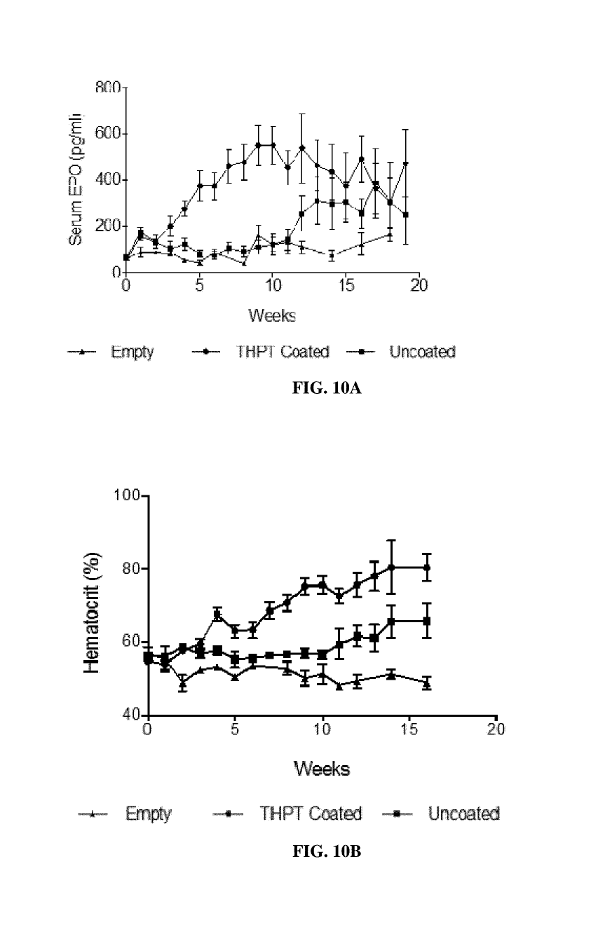

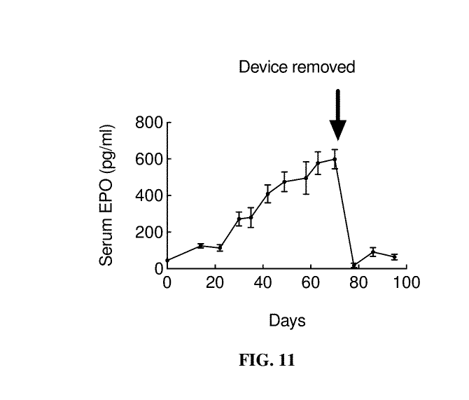

E9 coated macrodevices maintain grafted human cells (HEK) in C57B16 mice for over 18 weeks. Serum EPO levels of animals with THPT coated (n=21), uncoated (n=10) and empty devices (n=5) for a period of 19 weeks. Note that since a dividing cell line was used, it was observed that in uncoated devices the cells can repopulate the device after the fibrosis is over. In contrast, the THPT coated macrodevices show a steady increase in serum EPO (corresponds to increasing cell number) indicating minimal fibrosis and adverse reactions, FIGS. 10A and 10B.

Therapy can be reversed by removing the devices. THPT coated macrodevices loaded with epo-HEK cells were transplanted in IP space of C57BL/6 mice and were successfully retrieved intact after 75 days. The serum EPO returned to baseline within 4 days of surgery indicating the reversal of therapy, FIG. 11.

### Example 2

### Long-term Survival of Islets (Rat) in STZ-Diabetic C57BL6 Mice

Materials and methods

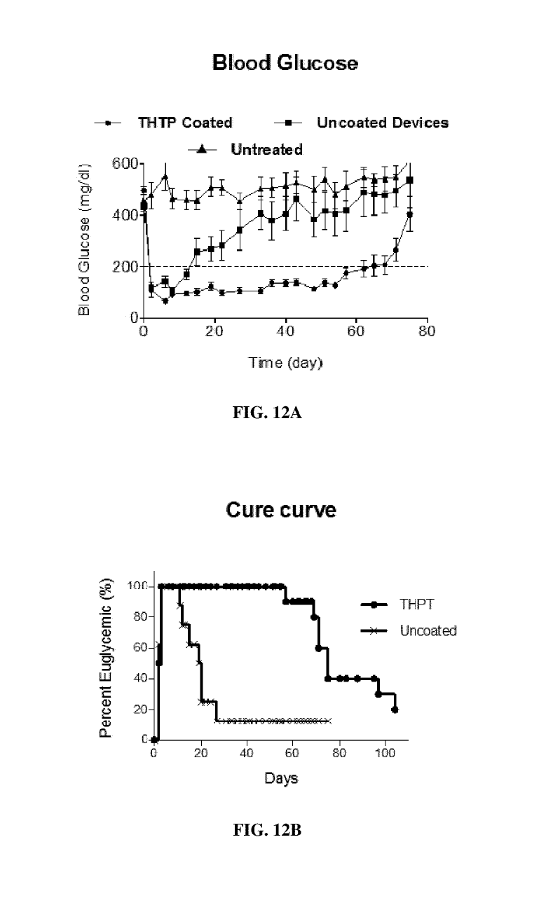

Rat islets were encapsulated in THPT (E9) coated macrodevices described in Example 1 and implanted in the IP space of STZ diabetic C57BL/6 mice. The blood glucose (BG) was monitored weekly, FIG. 12A.

Results

The BG measurements show that the THPT coated macrodevices cured mice for longer periods of time in comparison to uncoated macrodevices (n=5). A combined cure curve from two separate experiments (n=10 for THPT, n=8 uncoated) shows a median cure for 75 days in case of THPT devices and 19.5 days for uncoated macrodevices, FIG. 12B.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A macrodevice comprising a body having at least one or multiple compartments, wherein the one or multiple compartments comprise:
one or more side walls,
a top portion comprising a porous membrane attached to an edge of the one or more side walls,
a bottom portion, and
wherein outlines of the top and bottom portions are the same or different; and wherein the body has a largest dimension between 10 μm and 50 mm, inclusive.

2. The macrodevice of claim 1, wherein the one or multiple compartments comprise one or more cells, preferably cells that produce a therapeutically effective substance.

3. The macrodevice of claim 1, wherein the multiple compartments are arranged in a one-dimensional, two-dimensional array, or three-dimensional array.

4. The macrodevice of claim 1, further comprising one or more layers of a biocompatible component partially or completely coating a surface of the micro-fabricated body.

5. The macrodevice of claim 4, wherein the one or more layers of the biocompatible component are covalently and/or non-covalently bound to a surface of the macrodevice.

6. The macrodevice of claim 4, wherein the one or more layers comprise small molecules, polymers, or a combination thereof.

7. The macrodevice of claim 1, wherein the porous membrane has a pore size that allows diffusion of therapeutic agents, diagnostic agents, prophylactic agents, nutrients, oxygen, or combinations thereof, but inhibits host immune cells from entering the compartments.

8. The macrodevice of claim 1 having an overall length, X, an overall width, Y, and an overall height, Z, wherein each X, Y, and Z is independently an integer between 10 μm and 50 mm, inclusive, with the proviso that X and Y are selected such that X is always greater than Y.

9. The macrodevice of claim 1, wherein at least one compartment has a perpendicular distance of between 10 μm and 1 mm, inclusive, from the porous membrane to the bottom portion, preferably the perpendicular distance is about 0.25 mm.

10. The macrodevice of claim 2, further comprising integrated fluidic channels to access the compartments enclosing the cells.

11. The macrodevice of claim 1, wherein the porous membrane comprises pores having a pore size between 0.1 μm and 3.0 μm, inclusive.

12. The macrodevice of claim 1, wherein the micro-fabricated body and porous membrane independently comprise a material selected from the group consisting of polydimethoxysiloxane, medical grade silicone, polycarbonate, and polyurethane.

13. The macrodevice of claim 1, wherein the macrodevice has an oblong shape.

14. The macrodevice of claim 1, wherein the macrodevice has rounded corners.

15. The macrodevice of claim 4, wherein the biocompatible component comprises polymers, small molecules, or a combination thereof.

16. The macrodevice of claim 4, wherein the biocompatible component inhibits fibrosis of the macrodevice and allows improved functional viability of the encapsulated cells for at least five days compared to a control macrodevice that does not have the biocompatible component.

17. The macrodevice of claim 15, wherein the polymers are homopolymers, random co-polymers, block co-polymers, or a combination thereof.

18. The macrodevice of claim 15, wherein the polymers comprise monomeric subunits A, B, C, or combinations thereof, wherein:
each A is a zwitterionic monomer;
each B is a monomer having a reactive side chain; and
each C is independently a hydrophobic monomer, or a neutral hydrophilic monomer,
wherein the reactive side chain is d-R₁-Y;
wherein d is the point of covalent attachment of the reactive side chain to the backbone of the polymer;
wherein:
R₁ is alkyl, substituted alkyl, alkenyl, substituted alkenyl, alkynyl, substituted alkynyl, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), poly(lactic-co-glycolic acid), peptide, or polypeptide group;
Y is propane-1,3-dithiol, 1,2-dithiolan-3-yl, 1,2-dithiol-3-ylidene, amine, hydrogen, —SH, maleimide, aziridine, —N_{3,} —CN, acryloyl, acrylamide, —C(O)OR_{2,} —C(O)R_{3,} vinyl sulfone, —OH, cyanate, thiocyanate, isocyanate, isothiocyanate, alkoxysilane, vinyl silane, silicon hydride, —NR₄R_{5,} acetohydrazide, acyl azide, acyl halides, N-hydroxysuccinimide ester, sulfonyl chloride, glyoxal, epoxide, carbodiimides, aryl halides, imido ester;
wherein R_{2,} R_{4,} and R₅, are, independently, hydrogen, amino, hydroxyl, thiol, oxo, phosphate, or substituted or unsubstituted C₁-C₁₀ alkyl, C₁-C₁₀ alkylene, C₂-C₁₀ alkenyl, C₂-C₁₀ alkynyl, C₁-C₁₀ alkoxy, C₁-C₁₀ alkylamino, C₁-C₁₀ alkylthio, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, or substituted heterocyclic; and
wherein R₃ is hydrogen, amino, hydroxyl, thiol, oxo, phosphate, or substituted or unsubstituted C₁-C₁₀ alkyl, C₁-C₁₀ alkylene, C₂-C₁₀ alkenyl, C₂-C₁₀ alkynyl, C₁-C₁₀ alkoxy, C₁-C₁₀ alkylamino, C₁-C₁₀ alkylthio, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, or substituted heterocyclic.

19. The macrodevice of claim 18, wherein R₁ is -Aq-unsubstituted C₁-C₁₀ alkylene-Bq-unsubstituted C₁-C₁₀ alkylene-, Aq-unsubstituted C₁-C₁₀ alkylene-Bq-substituted C₁-C₁₀ alkylene-, -Aq-substituted C₁-C₁₀ alkylene-Bq-unsubstituted C₁-C₁₀ alkylene-, or Aq-substituted C₁-C₁₀ alkylene-Bq-substituted C₁-C₁₀ alkylene-, wherein Aq and Bq are independently —C(O)O—, —C(O)NH—, —OC(O)—, —NHC(O)—, —O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

20. The macrodevice of claim 18, wherein R₁ is —C(O)O-unsubstituted C₂ alkylene-NHC(O)-unsubstituted C₄ alkylene-, —C(O)O-unsubstituted C₂ alkylene-NHC(O)-substituted C₄ alkylene-, —C(O)O-substituted C₂ alkylene-NHC(O)-unsubstituted C₄ alkylene-, or —C(O)O-substituted C₂ alkylene-NHC(O)-substituted C₄ alkylene-.

21. The macrodevice of claim 18, wherein Y is Y is propane-1,3-dithiol, 1,2-dithiolan-3-yl, 1,2-dithiol-3-ylidene, amine, hydrogen, or —SH.

22. The macrodevice of claim 18, wherein the reactive side chain comprises a structure selected from the group consisting of

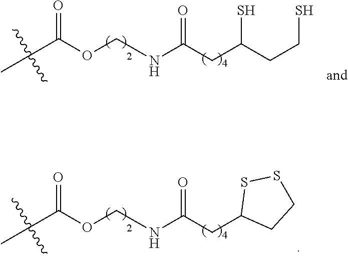

23. The macrodevice of claim 18, wherein the zwitterionic monomer subunit comprises a betaine, selected from the group consisting of carboxybetaine moiety, a sulfobetaine moiety, or a phosphoryl choline moiety.

24. The macrodevice of claim 18, wherein the zwitterionic moiety has a formula: 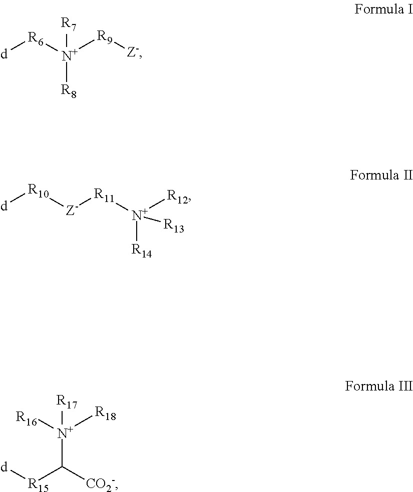
wherein d is the point of covalent attachment of the zwitterionic moiety to the backbone of the polymer;
Z is a carboxylate, phosphate, phosphonic, phosphanate, sulfate, sulfinic, or sulfonate; and
R₆-R₁₈are independently unsubstituted alkyl, substituted alkyl, unsubstituted alkenyl, substituted alkylene, unsubstituted alkylene, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group.

25. The macrodevice of claim 24, wherein R₆-R₁₈ are independently unsubstituted C₁-C₁₀ alkyl, substituted C₁-C₁₀ alkyl, unsubstituted C₁-C₁₀ alkenyl, substituted C₁-C₁₀ alkylene, unsubstituted C₁-C₁₀ alkylene, substituted C₁-C₁₀ alkenyl, unsubstituted C₁-C₁₀ alkynyl, substituted C₁-C₁₀ alkynyl, unsubstituted aryl, substituted aryl, unsubstituted heteroaryl, substituted heteroaryl, unsubstituted C₁-C₁₀ alkoxy, substituted C₁-C₁₀ alkoxy, unsubstituted aroxy, substituted aroxy, unsubstituted C₁-C₁₀ alkylthio, substituted C₁-C₁₀ alkylthio, unsubstituted arylthio, substituted arylthio, unsubstituted C₁-C₁₀ carbonyl, substituted C₁-C₁₀ carbonyl, unsubstituted C₁-C₁₀ carboxyl, substituted C₁-C₁₀ carboxyl, unsubstituted C₁-C₁₀ amino, substituted C₁-C₁₀ amino, unsubstituted C₁-C₁₀ amido, substituted C₁-C₁₀ amido, unsubstituted C₁-C₁₀ sulfonyl, substituted C₁-C₁₀ sulfonyl, unsubstituted C₁-C₁₀ sulfamoyl, substituted C₁-C₁₀ sulfamoyl, unsubstituted C₁-C₁₀ phosphonyl, substituted C₁-C₁₀ phosphonyl, unsubstituted polyaryl, substituted polyaryl, unsubstituted C₃-C₂₀ cyclic, substituted C₃-C₂₀ cyclic, unsubstituted C₃-C₂₀ heterocyclic, or substituted C₃-C₂₀ heterocyclic.

26. The macrodevice of claim 24, wherein R₆, R₉, R_{10,} R₁₁ and R_{15,} are independently unsubstituted C₁-C₅ alkyl, substituted C₁-C₅ alkyl, substituted C₁-C₅ alkylene, or unsubstituted C₁-C₅ alkylene, C₁-C₅ alkoxy, substituted C₁-C₅ alkoxy, unsubstituted aroxy, substituted aroxy, unsubstituted C₁-C₅ alkylthio, substituted C₁-C₅ alkylthio, unsubstituted arylthio, substituted arylthio, unsubstituted C₁-C₅ carbonyl, substituted C₁-C₅ carbonyl, unsubstituted C₁-C₅ carboxyl, substituted C₁-C₅ carboxyl, unsubstituted C₁-C₅ amino, substituted C₁-C₅ amino, unsubstituted C₁-C₅ amido, substituted C₁-C₅ amido, unsubstituted C₁-C₅ sulfonyl, substituted C₁-C₅ sulfonyl, unsubstituted C₁-C₅ sulfamoyl, substituted C₁-C₅ sulfamoyl, unsubstituted C₁-C₅ phosphonyl, or substituted C₁-C₅ phosphonyl.

27. The macrodevice of claim 24, wherein R_{7,} R₈, R₁₂, R₁₃, R₁₄, R₁₆, R₁₇, and R₁₈, are independently hydrogen, unsubstituted C₁-C₅ alkyl, or substituted C₁-C₅ alkyl.

28. The macrodevice of claim 18, wherein the zwitterionic monomer comprises a moiety selected from the group consisting of

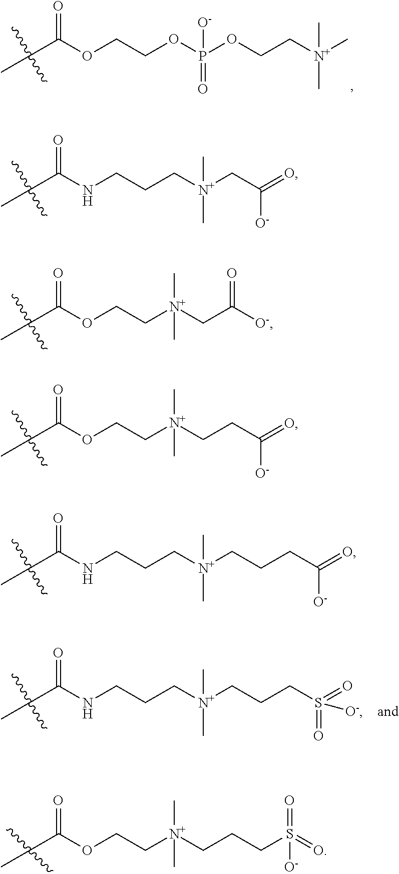

29. The macrodevice of claim 18, wherein at least one A is a first zwitterionic monomer and at least one B is a monomer with a reactive side chain.

30. The macrodevice of claim 18, wherein at least one C is a hydrophobic monomer comprising a hydrophobic side chain having the formula: 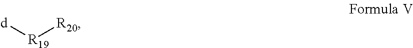
wherein d is the point of covalent attachment of the hydrophobic side chain to the backbone of the polymer; and
R₁₉ and R₂₀ independently unsubstituted alkyl, substituted alkyl, unsubstituted alkenyl, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, —O—, —S—, —NH—NHC(O)—, —N═N—, —N═CH—, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group.

31. The macrodevice of claim 30, wherein R₁₉ is —C(O)NH—, —C(O)O—, —NHC(O)—, —OC(O)—, —O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

32. The macrodevice of claim 30, wherein R₂₀ has the structure:
-Az-Bz-(—Cz)δ Formula VII
wherein δ is an integer between 0 and 10, inclusive;
Az is 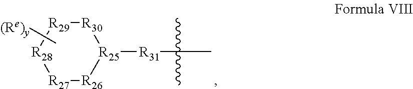
wherein R₃₁ in Az is —(CR₃₂R₃₂)ₚ—; p is an integer from 0 to 5; each R₃₂ is hydrogen, unsubstituted alkyl, or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R_{25,} R₂₆, R_{27,} R₂₈, R_{29,} and R₃₀ are independently C or N, wherein the bonds between adjacent R₂₅ to R₃₀ are double or single according to valency, and wherein R₂₅ to R₃₀ are bound to none, one, or two hydrogens according to valency, or Az is 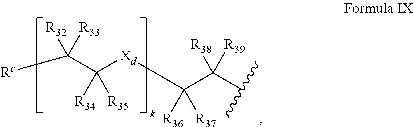
wherein R₃₂, R₃₃, R₃₄, R₃₅, R₃₆, R₃₇, R₃₈, and R₃₉ in Az are independently hydrogen, unsubstituted alkyl, substituted alkyl, unsubstituted alkenyl, substituted alkenyl, unsubstituted alkynyl, substituted alkynyl, unsubstituted phenyl, substituted phenyl, unsubstituted aryl, substituted aryl, unsubstituted heteroaryl, substituted heteroaryl, unsubstituted arylalkyl, substituted arylalkyl, unsubstituted alkoxy, substituted alkoxy, unsubstituted aroxy, substituted aroxy, unsubstituted carbonyl, substituted carbonyl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, substituted C₃-C₂₀ heterocyclic, poly(ethylene glycol), or poly(lactic-co-glycolic acid); k is an integer from 0 to 20; each X_{d} is independently absent, O, or S; and R^{c} is Bz;
wherein Bz is 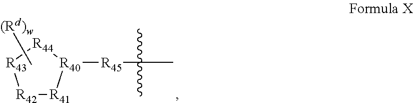
wherein R₄₅ in Bz is —(CR₄₆R₄₆)ₚ—; p is an integer from 0 to 5; each R₄₆ is hydrogen, unsubstituted alkyl, or substituted alkyl; each R^{d} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; w is an integer between 0 and 4, inclusive; each R_{40,} R_{41,} R_{42,} R_{43,} and R_{44,} are independently C or N, wherein the bonds between adjacent R₄₀ to R₄₄ are double or single according to valency, and wherein R₄₀ to R₄₄ are bound to none, one, or two hydrogens according to valency;
and wherein Cz is 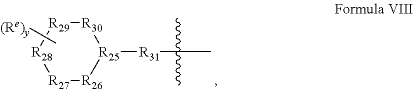
wherein R₃₁ in CZ is —(CR₃₂R₃₂)ₚ—or —(CR₃₂R₃₂)ₚ-X_{b} —(CR₃₂R₃₂)_{q}—; p and q are independently integers between 0 to 5, inclusive; each R₃₂ is hydrogen, unsubstituted alkyl, or substituted alkyl; X_{b} is absent, —O—, —S—, S(O)—, —S(O)₂—, or NR₄₇; R₄₇ is unsubstituted alkyl or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R_{25,} R₂₆, R_{27,} R₂₈, R_{29,} and R₃₀ are independently C or N, wherein the bonds between adjacent R₂₅ to R₃₀ are double or single according to valency, and wherein R₂₅ to R₃₀ are bound to none, one, or two hydrogens according to valency.

33. The macrodevice of claim 18, wherein the neutral hydrophilic monomer comprises a hydrophilic side chain having the formula: 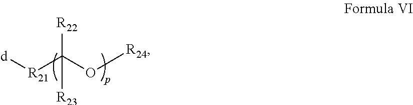
wherein, d is the point of covalent attachment of the neutral hydrophilic side chain to the backbone of the polymer;
p is an integer between 1 and 10,000, inclusive;
R₂₁ is unsubstituted alkyl, substituted alkyl, unsubstituted alkenyl, substituted alkenyl, unsubstituted alkynyl, substituted alkynyl, unsubstituted aryl, substituted aryl, unsubstituted heteroaryl, substituted heteroaryl, unsubstituted alkoxy, substituted alkoxy, unsubstituted aroxy, substituted aroxy, unsubstituted alkylthio, substituted alkylthio, unsubstituted arylthio, substituted arylthio, unsubstituted carbonyl, substituted carbonyl, unsubstituted carboxyl, substituted carboxyl, unsubstituted amino, substituted amino, unsubstituted amido, substituted amido, unsubstituted sulfonyl, substituted sulfonyl, unsubstituted sulfamoyl, substituted sulfamoyl, unsubstituted phosphonyl, substituted phosphonyl, unsubstituted polyaryl, substituted polyaryl, unsubstituted C₃-C₂₀ cyclic, substituted C₃-C₂₀ cyclic, unsubstituted C₃-C₂₀ heterocyclic, substituted C₃-C₂₀ heterocyclic, amino acid, poly(ethylene glycol), poly(lactic-co-glycolic acid), peptide, or polypeptide group; and
R_{22,} R_{23,} and R₂₄ are independently hydrogen, alkyl, substituted alkyl, alkenyl, substituted alkenyl, alkynyl, substituted alkynyl, aryl, substituted aryl, heteroaryl, substituted heteroaryl, alkoxy, aroxy, substituted aroxy, alkylthio, substituted alkylthio, arylthio, substituted arylthio, carbonyl, carboxyl, amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, or polypeptide group.

34. The macrodevice of claim 18, wherein the polymer comprises: 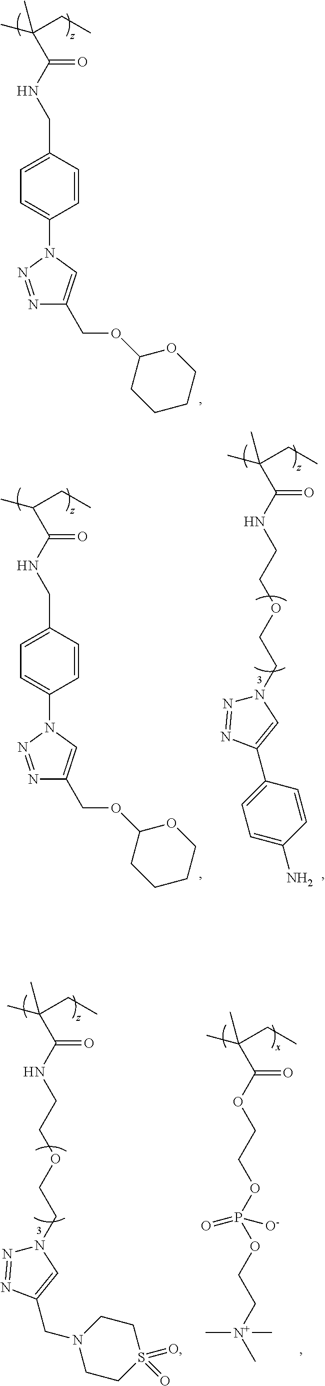 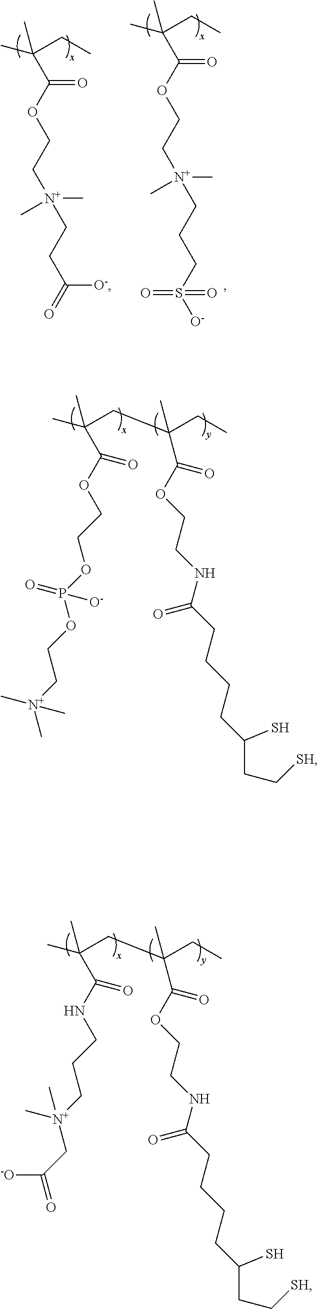 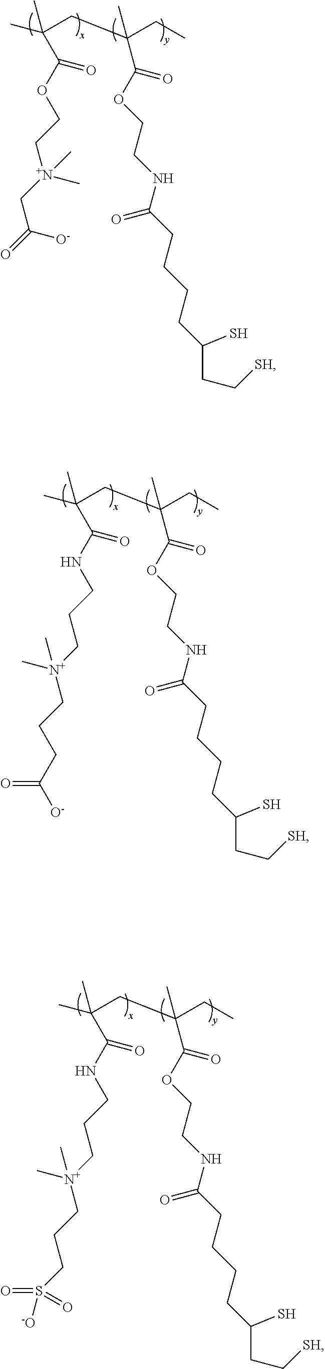 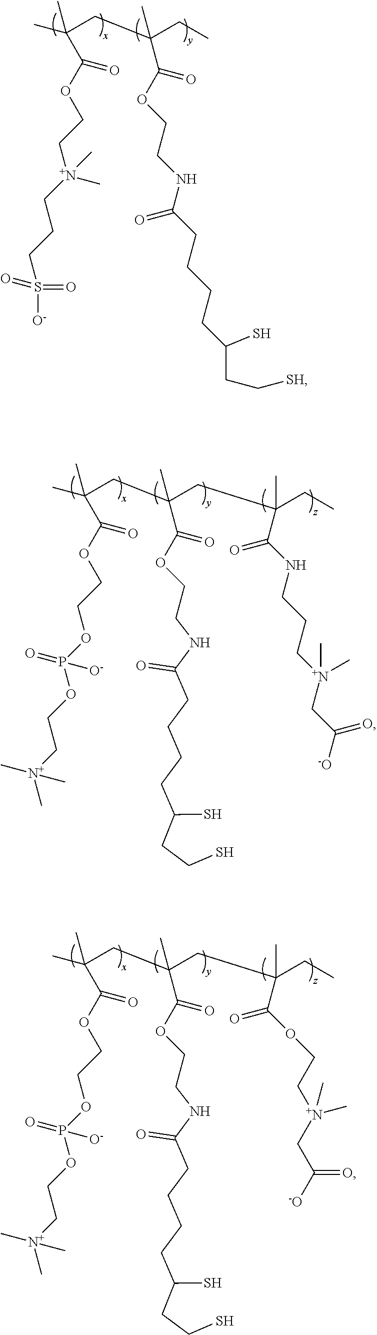 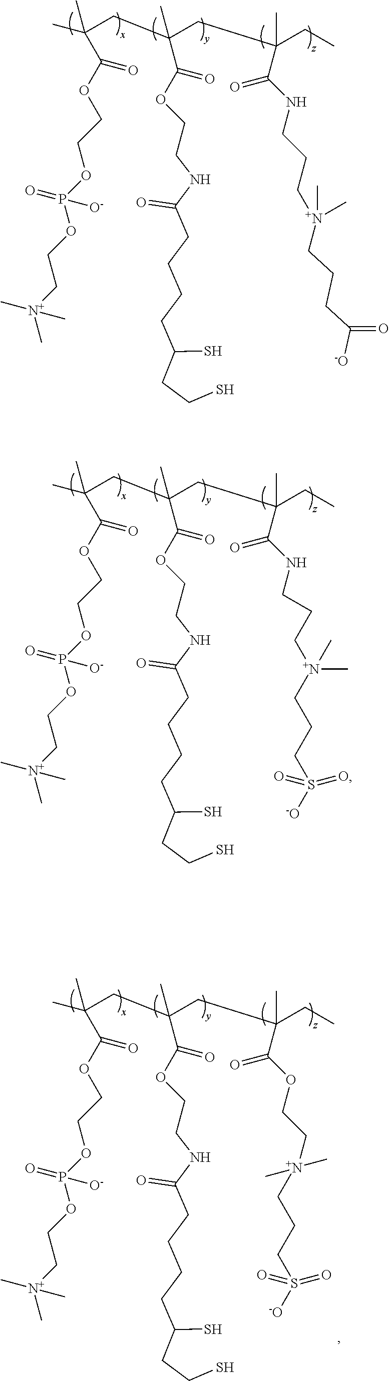 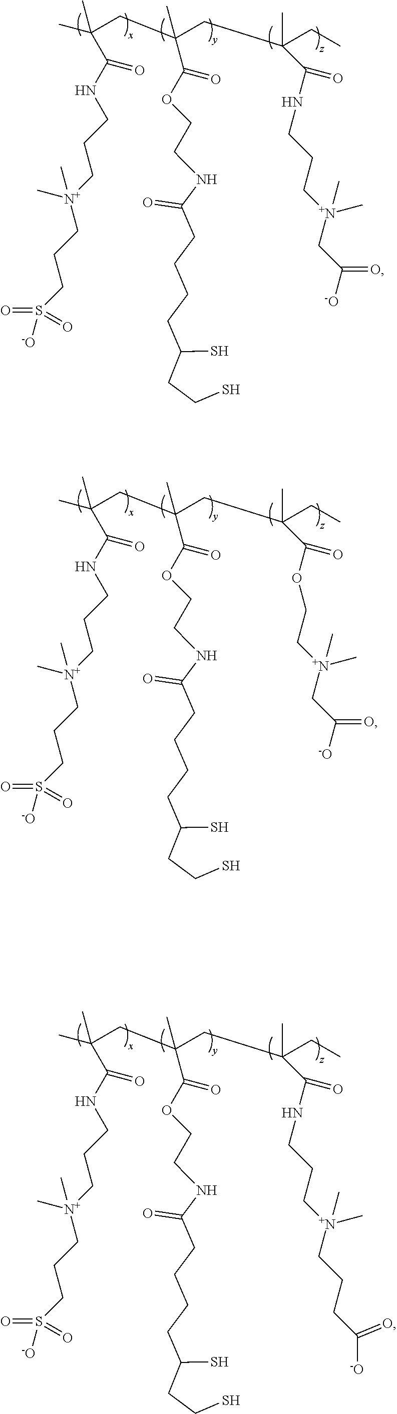 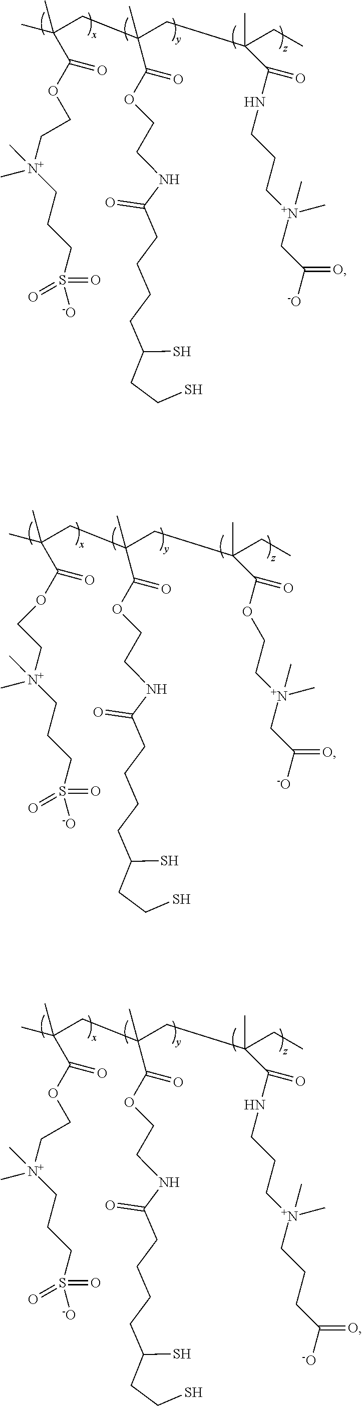 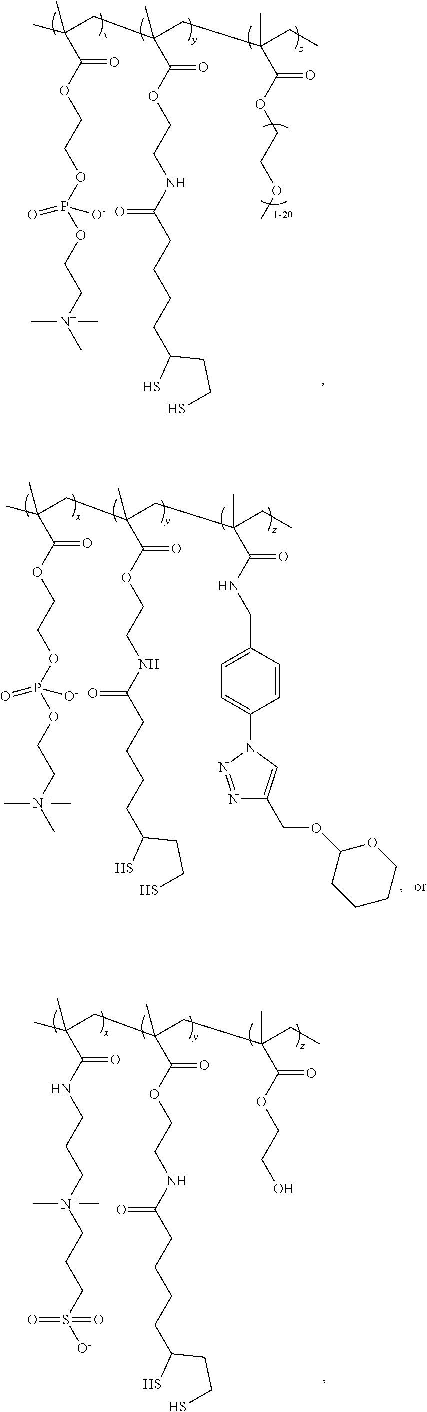
wherein, x and y are independently integers between 1 and 1000, inclusive, y is between 2 and 20, inclusive; and
z is between 0 and 1000, inclusive.

35. The macrodevice of claim 2, wherein the cells are selected from the group consisting of genetically engineered cells, stem cells, stem cell-derived cells, and pancreatic islets cells.

36. The macrodevice of claim 2, wherein the therapeutically effective substance comprises a protein.

37. The macrodevice of claim 2, wherein the therapeutically effective substance comprises a hormone, growth factor, enzyme, antibody, peptide, cytokine, or combinations thereof.

38. The macrodevice of claim 1, wherein the porous membrane is attached to the body via heat pressing, laser welding, chemical bonding, glues, or combinations thereof.

39. The macrodevice of claim 1, wherein the body is formed by microfabrication.
